(19) United States
(12) Patent Application Publication
(10) Pub. No.: US 2007/0298505 A1
(43) Pub. Date: Dec. 27, 2007

(54) Method of Screening Crude Oil for Low Molecular Weight Naphthenic Acids

(76) Inventors: Desmond Smith, Swindon (GB); Patrick Colin Smith, Greenford (GB); Sandra Ham, Newbury (GB)

Correspondence Address: REINHART BOERNER VAN DEUREN S.C.;ATTN: LINDA KASULKE, DOCKET COORDINATOR, 1000 NORTH WATER STREET, SUITE 2100, MILWAUKEE, WI, 53202, US

(21) Appl. No.: 11/766,636

(22) Filed: Jun. 21, 2007

(30) Foreign Application Priority Data
Jun. 21, 2006 (GB) 0612344.2

(51) Int. Cl. G01N 31/00

(52) U.S. Cl. 436/61

(57) ABSTRACT

A method for the quantitation of the naphthenic acid content of molecular weight less than 600 Daltons in a hydrocarbon composition, in which naphthenic acids are separated from the hydrocarbon composition and applied to a macroreticular ion-exchange resin. The resin is washed and the naphthenic acid-metal ion complexes are eluted from the resin. The number of naphthenic acid-metal ion complexes present in the eluate from the resin are then quantitated.

### CROSS REFERENCE TO RELATED APPLICATIONS

This application claims priority benefit under 35 U.S.C. §119(a) from Great Britain Patent Application No. GB 0612344.2 filed in the United Kingdom Intellectual Property Office on Jun. 21, 2006, the entirety of which is hereby incorporated herein by reference.

### BACKGROUND OF THE INVENTION

### Field of the Invention

The present invention relates generally to a method of screening crude oil to determine the amount of low molecular weight naphthenic acid present, and more particularly to a method providing a mechanism by which crude oil can be screened to assess quality and also purified to remove such impurities. References to screening crude oil include refinery product streams and desalter waters to evaluate fluid qualities in terms of fouling, corrosion, emulsion tendencies, and environmental concerns.

There are an increasing number of new oil reservoirs being discovered and developed in recent years that yield heavier oil types (i.e. oils with API gravities of between 15° to 25°). With sustained higher oil prices, such oilfields have become more viable. One common trait for these oils is that they show high total acid values and significant naphthenic acid contents. In refineries these oils are known as High Acid Crude (HAC) feedstocks. The actual Total Acid Number (TAN) values (milligrams of potassium hydroxide (KOH) per gram of oil) of these oils does not correlate at all with their risk of forming naphthenate solids or other soap types during reservoir fluid production in oilfields. These soap materials are an increasingly more common and very severe flow assurance problem being encountered worldwide [1 to 6]. For example, there are rare cases of 350 API oils of 0.10 TAN that can yield high amounts of naphthenate solid precipitates during production [5]. Alternative ways of defining the relative organic acid fraction of crude oils is a real need in the oil industry, for both the upstream and downstream industries.

Within the last five or more years there has been an increasing number of oilfields produced with varying amounts of naphthenic acids [1 to 6], or rather, the understanding in the oil industry has reached a level where people are now confident in publishing papers in this area. In truth, naphthenic acid problems have been experienced for at least fifty years in the oil industry, mainly manifesting themselves as stable emulsions and dirty disposal water problems in oilfields, e.g. in Southeast Asia such problems have been observed since the early 1960's. These problems were associated with the formation of strong surfactant properties of soaps of organic acids [1, 4, 5, 6 & 8 to 11]. It is noted that as existing crude oil reservoirs are becoming depleted, the newer reservoirs being discovered now are ones that in general are classed as immature reservoirs and are commonly biodegraded oils that have lost many components (e.g. alkanes and waxes), and thus the naphthenic acids are found to occur in increasing concentrations [2, 8, 9].

By definition a naphthenic acid is a monobasic carboxyl group attached to a saturated cycloaliphatic structure. It has been accepted in the oil industry that all organic acids in crude oil are called naphthenic acids. Naphthenic acids in crude oils are mixtures of low to high molecular weight organic acids. Such acids can be very water soluble to oil soluble depending on their molecular weight, on the process temperatures their parent fluids are subjected to, on the salinity and pH of their parent waters, and finally on their parent fluid processing pressures [1, 2, 5, & 6].

Naphthenic acid nomenclature was always known in peer groups as a misnomer as it implied unsaturation due to the “enic” in its name. Naphthenic acids were discovered over 100 years ago [1,2]. The old classification indicates that naphthenic acid is a monobasic carboxyl group attached to a saturated cycloaliphatic structure [1,2]. It has been stated that all organic acids in crude oil are called naphthenic acids but this may not be true [1,2]. This is simplistic, as acetic acid is not recognized as a naphthenic acid. Heavy Californian crude oils are known to yield naphthenic and naphtheno—aromatic acids, for example [13, 14, & 15].

Naphthenic acids in crude oils are mixtures of low to high molecular weights species varying from approximately 100 to greater than 1300 molecular weight (MW), as shown by various analyses, including mass spectrometric (MS) and Fourier Transform Ion Cyclotron Resonance (FTICR) analyses [6 & 7]. In the water phase, naphthenic acids can cause stable reverse emulsions. In the oil phase with residual water, these acids have the potential to react with a host of minerals, which can neutralize the acids to form insoluble salts.

The main reaction product found in actual oilfields is the calcium naphthenic acid soap, known to the oil industry as calcium naphthenate. Analyses of various actual soaps samples from oilfields around the globe indicates that they are often a mixture of one or more soaps (including calcium, magnesium, sodium, potassium, iron, and aluminum), with occluded silica/sand, mineral scales, iron hydroxides/oxides/sulphides, formation clay, mud residues (e.g. bentonites), flocculated asphaltenes, paraffin waxes, resins, and treating chemicals [5, 6, & 8]. These soaps and other occluded particles have been known under real conditions for a long time [16 & 17]. They adhere to oil-water interfaces and can become finely dispersed within the water itself. The oil industry to date has solved the emulsion problems caused by naphthenate soaps using acidic demulsifiers (called “pad busters”) or interface draw-offs to separate slop treatments [2 to 6 & 8]. The reverse emulsions in oils and in dirty oilfield water are being treated with water clarifiers, most of these being the cationic water clarifiers [5 & 8].

The naphthenic acids which form soaps and salts cause problems from refinery and oilfield crude inlet stock tanks creating slops; in desalters these appear as tight emulsions, lowering the desalting efficiencies. They can also react via double displacement to form high molecular weight foulants trapping other components in a tight matrix to foul heat exchangers, cause foaming in units, create interface pads, dirty disposal water, carry over of sodium, calcium, and other metals to bottom fractions and products. There is a range of other problems associated with naphthenic acids, including the initiation of gum and/or polymer formations, poor dewatering in product stock tanks, transfer of metals, and scale.

The normal method of evaluating crude oils to the refinery with special reference to refining problems involves a lot of wet chemistry, including asphaltenes content, ash & carbon residue contents, TAN ASTM D664, and distillation yield tests. Service companies can perform the high temperature fouling tests with and without antifoulants. Gas chromatography-mass spectrometry (GC-MS) is carried out to determine the range of naphthenic acids.

For the oilfield, the low molecular naphthenic acids and/or other carboxylic acids are not routinely determined, as no oilfield method exists for performing this analysis. The low molecular weight acids (LMWA's) are important in emulsion and desalting work in upstream production processes. A ranking of degree of emulsion or poor desalting efficiencies for upstream crude oil samples prior to the present invention does not exist at present.

Based on the main refinery laboratory tests, crude oil refining slates can be determined and blended depending on the GC-MS results to minimize refining problems of fouling and corrosion without lowering the economics of the refinery. The TAN test is a standard test and estimates total acids not only organic naphthenic acids. GC-MS resolves the naphthenic acids into hundreds of components, and not all components are identified as naphthenic acids. Although the refinery attempted to do this via GC-MS, there are still extensive corrosion, emulsion, and fouling problems in refineries which are not totally answered.

Corrosion via all acids, not only naphthenic acids, is a major problem in refineries from the preheat furnaces to the overheads on to the high temperature heat exchangers, e.g. the crude unit bottoms. Refiners have managed some of these corrosion problems by choice of certain stainless steels or titanium metallurgy and a chemical corrosion program. There is therefore a need for improvement in understanding the corrosion potentials of crude oils.

Fouling of processes occurs due to the inherent and potential kinetic cleanliness of the crude, the potential instability of the free radicals produced in heaters to form gums and polymers, and reactions of naphthenic acids at elevated temperatures with metals in the entrained water, mainly calcium and iron. The inherent fouling potential of the crude oil implicates several materials which can be included in the sales crude: particulate matter, clay, drilling and completion muds, bentonites, sand, metals as oxides and sulphides, and inorganic and organic scales.

The organic scale is mainly the naphthenate soap type, although other scale types have been identified, e.g. phosphorus and nitrogen containing types. Naphthenic acid is found in most crude oils at wide ranging values from parts per million to percentage levels. Naphthenic acids influence most of the common problems associated with fouling, corrosion, and emulsions in refining crude oils with TAN values from 0.5 upwards.

Naphthenic acids form the natural surfactant in crude oils and trap water. Reactions with alkali and alkaline earth metals give the well-known oil soluble/dispersible salt and soaps we recognize as present in crude oils. These compounds are difficult to remove as removal of calcium, sodium, or potassium from soap in a desalter requires breakage of the soap C—O-Metal bond. This is not possible unless the desalting chemical contains an acid, chosen from a mineral acid, phosphorus acids, acetic or sulphonic acid, or combinations thereof.

Inefficient removal of the metals means that some of the surfactant soap and a portion of LMWA's are transferred to the water phase as a reverse emulsion requiring water clarifiers. In addition to the transfer of soaps to the water in the desalter, some soaps reside in the crude oil leaving the desalter to the preheat furnaces, preheat exchangers (cause fouling and corrosion) and then to the crude distillation unit. Free LMWA naphthenic acids are very oil soluble at desalter temperatures whilst the high molecular weight acids (HMWA's) are present as particulate micelles finely dispersed and hydrogen bonded to water, ensuring their transport to the tower.

These equipment and units will be exposed to fouling as naphthenate soaps and naphthenic acids act as binders for the contaminants. A portion of the free naphthenic acids can react in these units and equipment to form soaps of the metals mentioned above. There are always free naphthenic acids remaining at the inlet of the crude unit to give a TAN value. The low molecular weight acids are distillable, fractionate into the main fractions, and can appear as emulsions in recycle drums, condensate knockout vessels, and in the overhead vapors.

Some naphthenic acids (both low molecular weight and high molecular weight) in the crude unit at the high inlet temperatures fracture to lower molecular weight naphthenic acids, a portion of which appear as volatile organic acids, giving a degree of unsaturation in bond breakage to the hydrocarbon fragments of this reaction. This unsaturation is the precursor to gum and olefin polymerization reactions.

Naphthenic acids and naphthenate soaps are natural surfactants, which can cause foaming in the crude units or other units. These can then be physically transported upwards into lower boiling fractions to impact the cleanliness of the product streams. Naphthenate soaps also transport the metal calcium, iron, sodium, etc. upwards to the trays, depositing scales in the units. These metals are found in the diesel and fuel oil streams, which affect their compliance with specifications.

These acids, by means of their surfactant properties and oxygen/hydroxyl groups in their molecules, are capable of trapping very fine water droplets via Hydrogen bonding and fine micelle, emulsion formation to cause some product streams to become hazy in stock tanks. The entrained water—surfactant in organic fuel fractions cause pumpability problems in the water separation index, modified (WSIM) test (ASTM D2550) for fuels. The products will be off specifications and require dehazing chemicals to settle and separate off the water or rerun for drying via diatomaceous earth or resins.

Thus, naphthenic acids and soaps affect fouling, corrosion, emulsions, desalting, and product quality, among the major negative influences to be experienced in the refineries. Low TAN's light crude oil supplies are becoming depleted, and thus there is a high proportion of naphthenic acid crude oils on world markets. A noticeable change is recorded in the quality of new reservoirs being discovered with respect to these types of naphthenic acid crude oils.

These new available crude oils with high TAN's are sold at a discount on world markets ranging from US$3 to US$30 a barrel depending on prevailing market values of crude oils, acid values, API gravities, and yield values. Thus, there is a need to determine the qualities of these crude oils in a refining environment to take advantage of a less expensive crude oil.

The design of typical production processes and the decreases in fluid pressures from wells to separation trains affect the bicarbonate, carbon dioxide, and pH equilibrium in produced waters. This provides a driving force for the formation of calcium naphthenate soaps [5, 6, 18, & 19]. These calcium naphthenate soaps (including contaminants), can deposit across the oil process system from wellheads, subsea flowlines, separators, chemelectrics, heat exchanger tubes to oil filters, and even within oil storage tanks. These deposits can congeal to hardened cement-like structures requiring frequent system shutdowns and physical cleanup periods costing millions of dollars in maintenance costs and lost production [5].

Calcium naphthenate dispersed in crude oils to refineries can sludge out in terminal tanks and cause fouling in lines, desalters, and heat exchanger units. In refineries, most analyses of the naphthenic acid fractions are important due to implications in the corrosion process [9, 17, 20, & 21]. Results are reported on the medium molecular weight (250 to 450 MW) naphthenic acids to enable prediction of overheads corrosion/neutralizer rates [22] and the amount of caustic and/or ammonia double decomposition required to combat naphthenic acids and soaps prior to desalters and distillation units. There is no distinction between high and low molecular weight naphthenic acids in refineries. Prior to the tetrameric naphthenic acids (ARN acids) discovery [7], mass spectrometric analyses were concentrating in the LMWA area of up to 320 MW [18].

In the water phase, naphthenic acids and their soaps have been known to cause stable reverse emulsions (i.e. oil droplets in a continuous water phase) in some production systems [5]. In the oil phase with residual water, these acids have the potential to react with a host of minerals, which are capable of neutralizing the acids. The main reaction product found in practice is the calcium naphthenic acid soap (the calcium salt of naphthenic acids). These often become insoluble salts and form solids material that can plug production systems, eventually causing system shutdowns [1 to 6]. Analysis of these collected soap solids has proven that calcium soap is often a major component. Soap agglomerations in wells and separation trains can generate a mixture of magnesium, sodium, potassium, iron, and aluminum soaps with occluded formation-derived sand, silt, and clays, mineral scales, iron scales, precipitated asphaltenes, resins, petroleum waxes, mud components, and treatment chemicals [5,6].

Several gas chromatography and combined expensive mass spectrometric analytical procedures have not so far been able to give acceptably accurate quantitative determinations of the precursor problematic acids contents in oils prone to generating these soaps. Other methods involve use of a pilot plant to determine the organic scale-soap probability under a combination of synthesized process conditions, typically using significant volumes of aged “stabilized” (degassed) crude oil samples [6].

A further development is the identification of the ARN acids, a newly defined set of naphthenic acids, typically of mass over charge (m/z) of 1230 amu (atomic mass units, or Daltons), which have been identified as being present in significant quantity in many oilfield naphthenate deposits and crude oils [5, 6, & 7]. It has been proposed that the specific acid types are one of the main precursor acids present in crude oils that must be available to form the calcium and other naphthenate soap solids [6 & 7]. The ARN acid structures have yet to be fully defined [7], but they are important acids and they do form a proportion of the acids isolated by the methodology described in this patent.

Previous and current techniques for analysis of naphthenic acids do not examine the relative amount of high molecular weight naphthenic acids. Expensive techniques are employed which are not valuable on a routine basis or practical in field conditions. Chromatographic techniques, including GC-MS [23] and mass spectroscopy (MS) [1,5], resolve the naphthenic acids into hundreds of peaks. Analysis of the naphthenate soaps by a Norwegian research group found a strange high molecular weight acid species, designated ARN acids, 1230+ MW, m/z, z=1, which is tetraprotic and also gives a 615 m−2H/z (z=2) signal [6 & 7]. These ARN acids were qualitatively detected as the major components in various naphthenate deposits. Previous researchers emphasized the generalized 250-450 MW areas for naphthenic acids [18 & 22]. Thus the ARN acid discovery was a revelation in the naphthenate soap story.

Many laboratory investigations have been performed on naphthenic acids and naphthenate solid soaps within the last five years. There are publications from Total [1, 2, & 10], Conoco Phillips and Statoil [7], ExxonMobil [22], Heriot-Watt University with Oil Plus [6], and university research students Brandl O. and Havre T. E. [19,24] of the Norwegian University of Science and Technology (NTNU), and Professor J. Sjoblom. Experimental work ranged from normal reaction of naphthenic acids with brine to form soaps [1] performed by Rousseau et al., emulsion studies looking at the effects of naphthenic acids [1,8], naphthenic acids on silica gel (25), near infrared work [26], and liquid chromatography—mass spectrometry (LC-MS) analysis, after anion exchange, the acid ion-exchange resins (IER) method (10), to name a few. None of these have correlated a ranking table or prediction of good oils versus bad oils with respect to their potential to form naphthenate soaps. The ARN acids discovery [7] is the building block to further work and presents the opportunity to use this, in a more realistic methodology, for problematic naphthenate soap predictions.

When evaluating literature search results for most published approaches to naphthenic acids quantification or isolation, it can be seen that there is a practical problem to be solved with respect to the significance of the amount of the naphthenate precursor ARN-type acids present in oils, that would lead to actual naphthenate soap deposition problems within oilfield production systems. One major question is how much of these cyclic acids would need to be present in a crude oil for that oil to be designated a problematic naphthenate-forming oil.

The very instrumental reliant LC-MS, electrospray mass spectrometry (ES_MS), and GC-MS techniques employed thus far (start of 2006) are not ideal for routine on-site work in oilfield laboratories [6]. There are no published results on the analytical ranking of the special properties of crude oils relative to known oils with problematic naphthenate soap formation tendency and to oils with no naphthenate soap formation tendency.

There is therefore a need for a process that overcomes the problems inherent in the methods used to date to identify low molecular weight species of naphthenic acids.

### SUMMARY OF THE INVENTION

According to a first aspect of the invention, there is provided a method for the quantitation of the naphthenic acid content of molecular weight less than 600 Daltons in a hydrocarbon composition, comprising: 1. separating naphthenic acids of molecular weight less than 600 Daltons from the hydrocarbon composition; 2. applying the separated naphthenic acids of molecular weight of less than 600 Daltons to a macroreticular ion-exchange resin additionally comprising transition metal ions coordinated with an amine or ammonia in which the metal ion amine/ammonia coordinated complexes are bound to the surface of the resin; 3. washing the resin; 4. eluting the naphthenic acid-metal ion complexes from the resin; and 5. quantitating the number of naphthenic acid-metal ion complexes present in the eluate from the resin.

The present invention therefore provides new methods in analyzing the crude oils and product streams in an oilfield and/or a refinery. The methods involve an innovative technique of separation of high and low molecular weight ranges of naphthenic acids. The high molecular weight (600 to 1300 MW) naphthenic acids have been found to be the major acid components of soaps in the foulant materials. The low molecular weight (LMW) naphthenic acids (less than 600 MW) are recognized to cause emulsion and corrosion problems in crude units, while the high molecular weight non-distillable naphthenic acids (HMWA's) and high molecular weight (HMW) soaps in the residual crude bottoms cause fouling and severe corrosion in bottoms heat exchangers. Methods for screening a hydrocarbon-containing sample for high molecular weight naphthenic acids are described in copending Great Britain Patent Application No. GB 0606387.9, filed on Mar. 30, 2006, and in corresponding U.S. patent application Ser. No. 11/731,186, filed on Mar. 30, 2007, which patent applications are hereby incorporated herein by reference.

This technique holds great potential as a screening tool for oil and refinery fractions, for new fields, refinery crude oil slates, and product streams. The method can be used on current refinery and production operations for troubleshooting present problems.

In its broadest sense, the naphthenic acid content of a hydrocarbon composition is a reflection of the content of all of the organic acids present in the sample, i.e. carboxylic acids. The term naphthenic acid may also be used more narrowly to describe certain specific organic acid components of crude oil as follows.

Single and multiple fused cyclopentane and cyclohexane rings where the carboxylic acid group is attached to an aliphatic side chain or to a cycloaliphatic ring may be described as naphthenic acids.

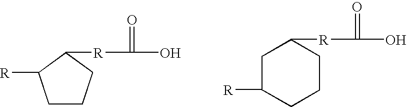

The American Petroleum Institute (API) definition for naphthenic acids includes structures with single and multiple fused cyclopentane and cyclohexane rings as illustrated in the diagram below, where the carboxyl group may be attached either to the aliphatic side chain (preferably) or to the cycloaliphatic ring (API 2003).

Based on these assumptions, it is possible to represent naphthenic acids by the formula CₙH_{2n+Z}O₂, where n is the carbon number and Z represents a homologous series. Z can be 0 or a negative even integer (−2, −4, etc.), and reflects the hydrogen loss or deficiency which occurs when a ring is included in the acid structure. Thus, Z equal to −2 indicates one cycloaliphatic ring, Z equal to −4 indicates two cycloaliphatic rings, etc. More than one isomer will typically exist for a given Z group. R may be any alkyl, or an alkenyl group which may be linear or a branched chain, or a ring or a connected system of rings, optionally substituted with O, N, or S, and may further may comprise saturated or unsaturated bonds.

As noted above, the term naphthenic acid may also used to identify all of the carboxylic acid content of a crude oil which can include alkyl substituted acyclics (including “fatty” acids), aromatic acids, carbazoles, and isoprenoid acids. More complex acid structures with two, three, and even four carboxylic groups (tetrameric acids) have been identified as well as structures containing heteroatoms (O, O₄, S, OS, O₂S, O₃S, N, NO, NO₂, N₂O) have been found in certain crude oils.

The specific acids may be tetrameric acids, in the molecular weight range of 1227 to 1235 Daltons. The acids homologous series corresponds to empirical formula of C₈₀H₁₃₈O₈, C₈₀H₁₄₀O₈, C₈₀H₁₄₂O₈, C₈₀H₁₄₄O₈, and C₈₀H₁₄₆O₈ with double bond equivalences (DBE) ranging from twelve to eight, indicating eight to four rings in the hydrocarbon skeleton, respectively.

References to naphthenic acid include naphthenate and vice versa unless the context clearly specifies otherwise.

Hydrocarbon compositions, which can be analyzed by a method of the invention, include crude oil, or partially purified crude oil, or an oil or substance obtained from crude oil following subsequent crude oil distillation, for example petroleum, kerosene, or paraffin. The method may be practiced on samples obtained from crude oil directly, or from sludges, oil deposits, oil emulsions, or tars which have been prepared for sample analysis. The method covers such organic acids as can be extracted from “raw” petroleum crude oil liquid samples as received after extraction (either dry or water containing), as well as acids extracted from oilfield production system deposits, scales, sludges, and emulsions, and including acids extracted from petroleum fractions of crude oils.

The crude oil may be a raw extract from a ground reservoir of oil following extraction, or it may be present in a refinery product stream, such as a distillate, fraction, or other residue from a process unit. The hydrocarbon composition may also be dispersed in water. Methods of the invention are therefore applicable to the analysis of waste water from a refinery where the hydrocarbon composition is dispersed in the water.

Preparation for sample analysis may include appropriate steps to remove particulate and/or solid matter, excess water, or other impurities. Excess water may be removed by a process of alternate heating and cooling of the sample, followed by centrifugation to remove the water. Alternatively, the water may be removed manually. The heating process may be carried out in an inert atmosphere, e.g. under nitrogen or helium or other inert gases.

The separation of naphthenic acid from the hydrocarbon composition may be achieved by means of a process such as dialysis. A dialysis procedure uses a membrane selected to be permeable to certain substances so as to permit a separation of the substance of interest from the remainder of the substances present. In the present case, the membrane may be permeable to low molecular weight naphthenic acids of molecular weight less than 600 Daltons to diffuse across the dialysis membrane. A preferred dialysis procedure is the closed sack distillative reflux dialysis method, which accomplishes extraction by dialysis under refluxing solvents. In such a method, the sample may be placed in a membrane dialysis sack (a.k.a. a finger cot). Sack/finger cot details are as follows. The supplier is DEK Northern Europe, Granby Industrial Estate, Weymouth, Dorset DT4 9TH, UK, Part No. 173035. The Sack/finger cot is made of anti-static latex ISO 5 (Class 100 7C), with the proprietary anti-static agent yielding average surface resistance of 5×10 12 Ohms Per square unit. It is washed, powder-free, 3 ml latex cleanroom processed and packaged in ESD safe inner bags and covering the small, medium, large, and extra large sizes (Product Numbers PN-173033, 173034, 173035, and 173057, respectively). During the distillation phase of the procedure, the membrane sacks may suitably be placed into a holder or a basket.

At the conclusion of the dialysis procedure, the solvent dialysate surrounding the dialysis sack is removed for sample preparation prior to application to the macroreticular ion-exchange resin for analysis. The sample may be further concentrated, or otherwise prepared for application onto the resin. The sample may be dissolved and volumetrically prepared in organic solvents.

The dialysis residue containing the higher molecular weight naphthenic acid content is then removed (and may be itself used for separate application to another macroreticular ion-exchange resin for analysis of the content of high molecular weight species in the sample).

Alternatively, the first step of separating naphthenic acid from the hydrocarbon composition and the second step of applying the separated naphthenic acid to the ion-exchange resin may be combined by direct addition of the hydrocarbon composition to the resin.

Suitable solvents may be a straight chain or branched C₁-C₁₀ alkane. The term C₁-C₁₀ includes the groups methyl, ethyl, propyl, butyl, pentyl, hexyl, heptyl, octyl, nonyl, and decyl. The alkane may be pentane, hexane, and/or heptane. Preferably, hexane may be used as the dialysis extraction solvent.

Suitably, the resin is packed into the form of a column so as to assist the analysis of the sample. While the resin is packed into the form of a column, the sample is loaded onto the column at one end.

The macroreticular ion-exchange resin may be a macroporous ion-exchange resin, suitably composed of a polymeric material. The polymeric material may be optionally cross-linked. For example, a suitable macro-reticular resin may be composed of cross-linked styrene divinylbenzene copolymers. A preferred type of such cross-linked styrene divinylbenzene copolymer macroreticular resins is Amberlyst® 15 and/or Amberlyst® A31 (available from Rohm & Haas Company, the owners of the Amberlyst trademark).

The macroreticular resin is modified in that it comprises transition metal ions coordinated with an amine or ammonia in which the metal ion amine/ammonia coordinated complexes are bound to the surface of the resin. Suitably, the transition metal ion is supplied in the form of a water-soluble salt, which can be subjected to admixture with the amine or ammonia to co-ordinate the transition metal ion. Suitable water-soluble salts include, but are not limited to, sulphate, nitrate, carbonate, and halide (i.e. fluoride, chloride, bromide, iodide). The resultant complex can be formed in situ on the column while the amine or ammonia is already bound to the column.

The transition metal ion may be one or more the following: a Period 4 transition metal ion, a Period 5 transition metal ion, a Period 6 transition metal ion, or a Period 7 transition metal ion. Alternatively, the transition metal ion may be a Group 3 (III B), Group 4 (IV B), Group 5 (V B), Group 6 (VI B), Group 7 (VII B), Group 8 (VIII B), Group 9 (VIII B), Group 10 (VIII B), Group 11 (I B), or Group 12 (II B) transition metal ion.

Period 4 transition metal ions include: Scandium (Sc), Titanium (Ti), Vanadium (V), Chromium (Cr), Manganese (Mn), Iron (Fe), Cobalt (Co), Nickel (Ni), Copper (Cu), and Zinc (Zn). Period 5 transition metal ions include: Yttrium (Y), Zirconium (Zr), Niobium (Nb), Molybdenum (Mo), Ruthenium (Ru), Rhodium (Rh), Palladium (Pd), Silver (Ag), and Cadmium (Cd). Period 6 transition metal ions include: Lutetium (Lu), Hafnium (Hf), Tantalum (Ta), Tungsten (W), Rhenium (Re), Osmium (Os), Iridium (Ir), Platinum (Pt), and Gold (Au).

Group 3 (III B) transition metal ions include Scandium (Sc), Yttrium (Y), and Lutetium (Lu). Group 4 (IV B) transition metal ions include Titanium (Ti), Zirconium (Zr), and Hafnium (Hf). Group 5 (V B) transition metal ions include Vanadium (V), Niobium (Nb), and Tantalum (Ta). Group 6 (VI B) transition metal ions include Chromium (Cr), Molybdenum (Mo), and Tungsten (W). Group 7 (VII B) transition metal ions include Manganese (Mn) and Rhenium (Re). Group 8 (VIII B) transition metal ions include Iron (Fe), Ruthenium (Ru), and Osmium (Os). Group 9 (VIII B) transition metal ions include Cobalt (Co), Rhodium (Rh), and Iridium (Ir). Group 10 (VIII B) transition metal ions include Nickel (Ni), Palladium (Pd), and Platinum (Pt). Group 11 (I B) transition metal ions include Copper (Cu), Silver (Ag), and Gold (Au) Group 12 (II B) transition metal ions include Zinc (Zn) and Cadmium (Cd).

Preferred transition metal ions may include metal ions selected from the group consisting of Copper (Cu), Silver (Ag), Nickel (Ni), Cobalt (Co), Iron (Fe), Manganese (Mn), Molybdenum (Mo), Chromium (Cr), and Platinum (Pt). More particularly preferred transition metal ions may be Copper (Cu), Nickel (Ni), or Molybdenum (Mo).

In one embodiment of the invention, the transition metal ion is not added to the resin (or resin prepared in the form of a column), but rather the complex is prepared on the resin or resin column, so the reaction to form the complex takes place on the resin or resin column.

The transition metal ion is coordinated with an amine or ammonia. The transition metal ion is suitably adsorbed on to the column and the amine or ammonia is then suitably fixed on to the metal to form the appropriate metal/ammonium or metal/amine complex in situ. For example, where the metal is copper, the use of ammonia will lead to the formation of the corresponding cuprammonium complex and the use of an amine will lead to the formation of the appropriate cupramine complex.

The amine may be an amine of general formula: 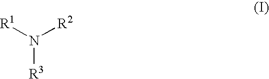
in which R¹, R² and R³ may each independently be Hydrogen (H), or a C₁-C₆ straight chain or branched alkyl group, optionally substituted with Oxygen (O). The term C₁-C₆ includes the groups methyl, ethyl, propyl, butyl, pentyl, and hexyl.

Where one or more of R¹, R² and R³ are an alkoxy group it may comprise from 1 to 14 alkoxylated groups, selected from ethylene oxide, propylene oxide, or butylene oxide and mixtures thereof.

The amine may be a linear, branched chain or fused-ring system amine. For a fused-ring system amine, either R¹ and R², or R² and R³, or R³ and R¹ can be joined together to form a ring which may be saturated or unsaturated.

The amine may be a primary, a secondary, or a tertiary amine. Suitable amines include methylamine (CH₃NH₂), ethylamine (C₂H₅NH₂), ethylene diamine (NH₂CH₂CH₂NH₂), pyridine, morpholine, cyclohexamine, or benzylamine. Where all groups R are Hydrogen, the general formula will represent ammonia.

A coordinated complex of the transition metal ion and the amine of general formula (I) or ammonia may be formed in situ on the macro-reticular resin by adsorption entrapment. Macroreticular resins, e.g. in the form of a macroreticular resin column, are specifically designed to trap metal ions.

After loading the sample onto the resin or resin column, it is suitably allowed to equilibrate to permit the formation of naphthenic acid complexes with the bound transition metal ion amine/ammonia complex. After a sufficient period of time has elapsed, the resin or resin column is then washed to remove an unbound sample.

The column may be washed with solvents such as xylene and/or heptane which may assist in removal of heavy oil residues. Xylene and/or toluene may also be used for this purpose. A final wash of isopropanol may be used to swell the resin to assist in washing the column of excess residues.

Washing the resin or resin column may therefore comprise one or more of the following steps: 1. wash with a xylene/heptane mixture; 2. wash with a xylene/toluene mixture; and 3. wash with an isopropanol/water mixture.

Suitable mixtures of the wash solvents may be as follows: heptane/xylene in the ratio of from 0:90 to 100:10, most suitably of from 40:50 to 60:50, and preferably of about 50:50.

Eluting the naphthenic acid-metal ion complexes may be achieved with a suitable eluant selected from the group consisting of C₆-aryl, straight chain or branched C₁-C₆ alkyl alcohol or C₆-aryl alcohol, ammonia, and straight-chain or branched C₁-C₆ alkylamine and mixtures thereof.

The term C₁-C₁₀ includes the groups methyl, ethyl, propyl, butyl, pentyl, hexyl, heptyl, octyl, nonyl, and decyl.

The C₁-C₆ alkylamine includes primary, secondary, and/or tertiary amines. The C₁-C₆ alkyl alcohol includes primary, secondary, and/or tertiary alkyl alcohols. C₆-aryl alcohol includes phenol.

Preferably, the straight chain or branched C₁-C₆ alkyl alcohol may be methanol, ethanol, and/or isopropanol. Preferably, the C₁-C₁₀ alkane is n-heptane.

The straight chain or branched C₁-C₆ alkyl alcohol may be formulated as an aqueous solution or may itself be used. The straight chain or branched C₁-C₆ alkyl amine and/or ammonia may be formulated as an aqueous solution or as a solution of ammonia in a straight chain or branched C₁-C₆ alkyl alcohol as alcoholic ammonia or amine. An example of this is methanolic ammonia.

Suitably, the proportion of C₁-C₆ alkyl alcohol to ammonia or amine may be in the ratio of from 95:5 to 80:20.

Following the elution of the complexed naphthenic acid-ammonia or amine coordinated transition metal ion, the eluate may be concentrated by evaporation of the elution solvents. The concentration of the same may be achieved by evaporation under an inert gas atmosphere (e.g. nitrogen or other inert gases like helium), lyophilisation, or by any other generally suitable means.

Quantitation of the concentration of naphthenic acid-metal ion complexes present in the eluate may be achieved by measuring the absorbance of complexed naphthenic acid-ammonia or amine coordinated transition metal ions present in the sample and correlating this absorbance from a calibration graph. The absorbance can be correlated to the concentration of the complexes present by using known standards as controls.

The formation of the complex yields an ion complex with a characteristic ultraviolet radiation (UV) absorption signal by virtue of the coordinated transition metal ion present in the complex. Quantitation of the amount of naphthenic acid in a sample may therefore be achieved using an assessment of the UV absorption of a sample by spectrophotometry techniques compared to known reference samples containing defined amounts of naphthenic acid, including samples where naphthenic acid is absent. Suitably, UV absorption measurement may be made at 215 nanometers to 225 nanometers, preferably at 218 nanometers to 222 nanometers.

Quantitation could also take place using means to detect the formation of radioactive complexes or complexes that give rise to fluorescent products.

The present invention therefore provides a newly developed screening method for isolation of and fingerprinting of a diagnostic family of low molecular weight naphthenic acids implicated in naphthenate soap material formation in oil production plants and in refinery facilities.

In a preferred embodiment of the invention, a method for the quantitation of naphthenic acid content in a hydrocarbon sample may include the steps of: 1. separating and purifying low molecular weight naphthenic acids from crude oil samples, e.g. by dialysis, including the substeps of a. using a dialysis membrane sack in the separation of the low molecular weight naphthenic acids fraction from raw crude oil samples, petroleum fractions, and deposits containing naphthenate soap (solids, soap-emulsions, and sludges); and b. optionally using a metal mesh basket of specific dimensions to protect the dialysis membrane sacks from bursting during distillative reflux dialysis extraction of test samples, with a basket holder being used for multiple samples to be run; 2. selecting a transition metal from the transition metal series; 3. preparing a macroreticular resin in a transition metal form with a ligand as described above and forming resin as a column; 4. applying the sample to the column; 5. washing the column with a heptane/xylene wash, or mixtures thereof, and subsequently washing it to remove excess uncomplexed sample with isopropanol-water mixtures, and then eluting the complexed naphthenic acid-coordinated ammonia/amine transition metal ion with an alcoholic amine solution; 6. identifying the ultraviolet (UV) absorption lambda max wavelength of metal coordinated naphthenic acid complexes from samples under test, with reference to known copper naphthenates (pure standards) and actual field naphthenate deposits acid fraction extracts, converted to the metal coordinated ammonium/amine forms, by elution through the macroreticular resin column; 7. identifying the optimum and maximum absorbance values required for metal coordinated naphthenic acid complexes, from samples under test, such that the absorbance remains within an acceptable lambda max range for the calibration graph; 8. identifying that the dilution of high absorbances obtained from the metal coordinated naphthenic acid complexes meet with the maximum absorbance value recommended and that this dilution is linear, including the substep of: a. establishing a calibration procedure using low molecular weight naphthenic acids extracted from actual samples of naphthenate soap deposits/sludges and crude oil emulsions recovered from actual oilfield production and refinery processes; 9. identifying the minimum volume of colored, metal coordinated naphthenic acid complexes required for UV analysis and for UV fingerprint testing and identification of the minimum detectable naphthenic acids amount (Limit of Detection, LOD) in test solution, which is 0.25 mg in this method for LMWA's; 10. quantifying the amount of low molecular weight naphthenic acids (LMWA) obtained in the dialysate residue of test samples (e.g. crude oils and sludges) relative to known LMWA fractions extracted from actual oilfield deposit samples; 11. normalization the percentage weight LMWA fraction in the dialysate residue of test samples back to the original crude oil; and 12. normalizing the percentage weight LMWA fraction of stabilized crude oil samples with previously unknown percentage weight LMWA fraction/problematic soaps quality to the known problematic crude oils.

Step 1.a. may comprise distilling off the dialysate solvent in order to achieve separation of low molecular weight naphthenic acids in the dialysate residue. In other embodiments, the sample may be added directly to the resin or resin prepared in the form of a column.

The UV fingerprinting method of crude oils allows the milligram content of LMWA to be determined from the calibration graph and this can then be expressed as the percentage weight LMWA fraction of each oil under test. This is used to screen oils by comparing them to blank oils that do not form naphthenates in the oilfield and by comparing the results to crude oils known for having soap and emulsion problems during production, including oils that give calcium naphthenate deposition.

Preferably the sample for analysis may be a crude oil sample, for example impure crude oil from exploration/appraisal wells, drill stem tests, test separator samples, downhole high pressure live samples (e.g. Schlumberger/Expro SPS/MDT/RCI type samples) or refinery charge to the heat exchangers. The sample could be crude oil residues recovered from oil refinery operations/plants. The sample could be crude oil or naphthenate deposits found in upstream petroleum processes or from refinery operations.

In the separation procedure, where dialysis is used, it may be preferable to use a basket or a holder for the dialysis membrane, such as a metal basket. The metal basket may be constructed of stainless steel, which will not be oxidized or react with low molecular weight naphthenic (LMWA) acids.

Preferably, the transition metal is chosen from a water soluble salt of Copper, Nickel, or Molybdenum.

In the preparation of the coordinated transition metal ion, the nitrogen containing ligand is preferably chosen from ammonia, ethylamine, and ethylene diamine.

After loading the column and washing excess unbound transition metal ion and/or unreacted impurities from the sample, the elution solvent sequence can be chosen from a straight chain or branched C₁-C₁₀ alkane, a C₁-C₁₀ alkane and C₆ aromatic solvent mix, aqueous C₁-C₆ alcohol, neat C₁-C₆ alcohol, and C₁-C₆ alcoholic ammonia/C₁-C₆ amine.

The neat alcohol may be methanol. The alkane may be chosen from C5, C6 or C7 alkanes or mixtures thereof, e.g., pentane, hexane, and/or heptane. In the alcohol/amine mixture, the alcohol may be ethanol, methanol, or isopropanol, or mixtures thereof, but is preferably methanol.

In a laboratory scale version of the method, the resin may be prepared as a column, for example in a burette. The burette may have a physical resin bed dimension of 35 centimeters×10 millimeters, with a reservoir height of 15 centimeters. The resin may be Amberlyst® A15. In such embodiments, the sample dialysis weight may range between one to eight grams and the dialysis time may range between four to twelve hours. The preferred sample weight may be six grams. The preferred dialysis time may suitably be six hours.

Where dialysis is used in the separation of the sample prior to loading onto the resin, the dialysis reflux-extraction solvent may be pentane, hexane, or heptane, with hexane being preferred.

In a laboratory scaled version of the method, the charge of the material from dialysate on the burette sized column may range from 75 milligrams to 250 milligrams, with a preferred range of 75 milligrams to 150 milligrams. In such embodiments, the elution rate may range from 1.5 to 3 milliliters per minute, with a preferred rate of 2 milliliters per minute. In such embodiments, the volume of eluates to be used may be standardized at 100 milliliters volume. Glacial acetic acid or formic acid can be used in sample loadings to a maximum of 20 microliters on the burette sized columns.

After elution of the naphthenic acid complexes from the column, the alcohol or alcohol-ammonia solutions may be gently evaporated off to dryness under a nitrogen blanket.

The residues obtained from the evaporation of the alcohol or alcoholic ammonia may be collected and combined in a suitable amount of alcoholic ammonia solution for UV analysis starting at 3 milliliters and concentrated by evaporation to a residue.

Standard naphthenic acids may be obtained as follows from source hydrocarbons. Crude naphthenate soaps may be isolated from crude oil by a clean up step then followed by a dialysis extraction procedure. Warm heptane and ambient temperature xylene may be used in the clean up step. After the cleanup step, the crude naphthenate soap may then be treated to remove oil and residues/asphaltenes. The dialysate residue (DSR) after dialysis extraction may be treated with warm (60° C.) xylene-glacial acetic acid (1:1) extract solution, evaporated to dryness then re-dialyzed twice to give the purified field naphthenic acids on evaporation to a residue.

The analysis of the amount of naphthenic acid in a sample or a standard may be carried out by measuring the UV absorption of a sample, wherein the lambda max UV absorption is set at 218 manometers to 222 nanometers+/−2 nanometers. Preferably, the maximum absorbance at lambda max range of 218 manometers to 222 manometers is set at 2.5+/−0.5 A. The LMWA absorbances across the UV range may be measured on a UV-computer link and results may be normalized to those of known crudes, enabling prediction of problematic soaps formation.

The oil analysis method described in the present application involves a novel combination of analytical techniques, which concentrate up the high molecular weight acids fraction of any crude oil sample. While concentrating on actual crude oils samples with a view toward using this analysis technique to screen them for their acid fraction contents, it is equally possible to use this method on samples of sludge, solid deposits, treatment chemicals, drilling muds, distilled crude oil fractions, oil products, etc. These isolated acids with other high molecular weight components are then complexed on to a specific type of resin column for later elution and ultraviolet (UV) spectrophotometric analysis. Contaminants are washed off the column first, leaving behind the organic acids of interest. Results show a gradation of percentage complexed acids versus good and bad oils relative to probability of soap formation resulting in oil production and/or oil refining problems.

This technique holds great potential as an oil screening tool for new oilfields and can even be used on current production operations for troubleshooting present problems, i.e. with compartmentalized oil reservoirs. On a well-by-well basis, one can determine the wells that give fluids that will generate soaps and those that do not, allowing a map to be built up of fluids produced versus potential soap generation. The method procedure is relatively rapid, simple to learn and run, very accurate when compared to the other oil screening techniques currently available, and only a small quantity of the test oil sample is required for this test method.

Using such a method to rank oils with respect to their risk of causing soap problems, in upstream oilfield production systems and in downstream crude refining plants, is the basis of this patent application.

Methods in accordance with the present invention will enable the determination of the percentage weight low molecular weight naphthenic (% LMW) acids fraction of crude oils, leading to a classification fingerprint index. This index will assist in evaluating crude oils from new oilfield developments because, once analyzed, they can be classified with respect to their degree of risk for naphthenate soap/emulsion formation and corrosion tendencies, relative to the percentage of LMW naphthenic acids. This prediction of problematic naphthenate soap/emulsion formation is becoming critically important for new oilfield developments and the design and operation of production facilities, such as refineries. Operating companies need to know up front if a new oil will be a naphthenate soap former or not, since the associated flow assurance issues can be very severe and will heavily influence the facilities design [5]. In addition, such operating companies can look at their overall oilfield development plan and decide upon the various alternative measures they could take in controlling any naphthenate related production problems, from wells to the topsides export oil system. These options would include dilution of problem oils with oils that will not form naphthenates, dilution of the produced waters to negate naphthenate formation, injection of treatment chemicals for naphthenate soap control, employing up front high pressure separators, etc. [2 to 5 & 8]. This oil screening tool for naphthenates potential will accordingly have a big impact on the decisions made by various oilfield operating companies in the future.

The present invention has taken the analysis of heavy naphthenic acid soap precursors a step further by using a combination of exhaustive distillative-reflux and dialysis separation techniques, giving a concentration of high molecular weight naphthenic acids and then further reaction of the naphthenic acids by trans-metallation to metal ligand coordinated-ammonium naphthenates, on a macroreticular resin column. This leads to highly UV active transition metal-complexed compounds, which can be detected, with a suitable scanning UV spectrophotometer, down to the 1.25 milligram level, in actual test solutions.

The bulk concentration of polar compounds based upon molecular weight can be performed by a separation technique known as dialysis, which is here paired with a second separation phase using macroreticular resin. Normal column chromatography, using alumina and silica gel, separates materials by polarity strength, which is related to molecular weight. Separation by normal column work is not bulk separation in groups, but rather fine separation due to material polarities. Preparatory Thin Layer Chromatography (PTLC) gives the same types of separations at narrow MW ranges, but it is difficult to use TLC on crude oil samples, since alumina will react with the naphthenic acids.

Dialysis is normally used in the well-known medical fields in blood purification re kidney dialysis. References are available in the food industry for pumped dialysis in non-organic electrolytes [27]. This type of dialysis is not the closed sack distillative reflux dialysis, but rather is pumped dialysis or direct current dialysis at ambient conditions.

References also exist for the macroreticular resins, but they are mainly used in the environmental field. Silver is chemically and physically bonded to a macroporous resin (macroreticular), to enable it to scavenge out biocides from effluent waters [28]. No reference has been found, though, for the use of these macroreticular resins in their amino/ammonia-metal ligand form, in separation of naphthenic acids from crude oils, by column complexation and trans-metallation. Types of anion exchange resins have been used to separate naphthenic acids [10,29], but not in the area of direct metal ammonia-coordinated complexations of naphthenic acids from crude oils. This is one reason why the methodology of the patent application has not been tried before.

Based on the synopsis of the naphthenate soap problems discussed previously, there is a need for a less expensive, less time-consuming, more accurate, and commercially more cost-effective analytical technique for screening oil samples for naphthenate formation potential. This development work here has shown advances in the application of combined analytical techniques, including separation of a specific molecular weight range of organic acids from crude oil and complexation of that acid fraction, to give a method, which can be used in actual onsite oilfield laboratories, as well as in a central land-based laboratory.

The methods provide means for quantifying the unknown percentage LMWA in the dialysate residue, calculating the percentage non-HMWA's in the DR of refinery streams thereby giving a cleanliness index, determining inherent and potential fouling indices, quantifying the amount of naphthenic acids in desalter disposal water streams for environmental concerns, and/or determining the emulsion tendency of the crude oil at the desalter stage compared to a known emulsified crude oil.

The quantitative methods of the present invention enables the isolation of low molecular weight and high molecular weight naphthenic acids to such a degree that their concentration can be determined in units of milligrams (mg) acid per liter of oil (mg/l) and the results can be expressed as percentage weight (% wt) acid fraction of oil, henceforth known as percentage High/Low Molecular Weight Acid (% HMWA/LMWA) fraction. This specific group of LMW naphthenic acids that this method targets are known to be in part responsible for the formation of problematic calcium naphthenate soap solids deposition and/or in the formation of sodium or potassium soap emulsion problems in many oilfield production facilities and refinery systems worldwide.

The present invention therefore also provides methods applicable to crude oils, deposits, process disposal water, refinery product streams, and bottoms asphalts to determine the ratio of high molecular weight to the low molecular weight naphthenic acids.

Preferred features for the second and subsequent aspects of the invention are as for the first aspect mutatis mutandis.

### DESCRIPTION OF THE DRAWINGS

The invention will now be further described by way of reference to the following examples and figures which are provided for the purposes of illustration only and are not to be construed as being limiting on the invention. Reference is made to a number of figures in which:

FIG. 1 shows a graphical representation of the content of Table 2;

FIG. 2 shows a graphical representation of the content of Table 3;

FIG. 3 shows a graphical representation of the content of Table 4;

FIG. 4 shows a graphical representation of the content of Table 5;

FIG. 5 shows a graphical representation of the content of Table 6;

FIG. 6 shows a graphical representation of the content of Table 7;

FIG. 7 shows a graphical representation of the content of Table 8;

FIG. 8 shows a graphical representation of the content of Table 9;

FIG. 9 shows a graphical representation of the content of Table 10;

FIG. 10 shows a graphical representation of the content of Table 11; and

FIG. 11 shows a graphical representation of the content of Table 12.

### DETAILED DESCRIPTION OF THE PREFERRED EMBODIMENT

Definitions

HMWA is a term that means high molecular weight naphthenic acids (molecular weight greater than 600). LMWA is a term that means low molecular weight naphthenic acids (molecular weight less than 600). HAC is a term that means High Acid Crude feedstocks such as West African, San Joaquin Valley (SJV), and Venezuelan Crudes. BR is a term that means non-distillable residue. DSR is a term that means dialysate residue.

Methods

The naphthenate soap solids from the West African field were purified to remove asphaltenes, crude oil, resins, mineral matter and particulates to yield a cleaner HMW soap. The HMW acids were liberated, and mass spectroscopy, thin layer chromatography (TLC), and Infra Red Spectroscopy (IR) were performed on them. The MS spectra matched Oil Plus, Norwegian, and French research results indicate that we have collected the 600-1230 HMWA's for calibration. The TLC indicated that naphthenic acids were present similar to another set of HMWA's obtained from a North Sea naphthenate deposit.

An elution sequence was designed to wash off resins, oil, and asphaltenes from the column. A metal coordinated complex is formed which is soluble in methanolic ammonia solution. Resins, tars, and bitumens are not soluble in aqueous Methanol-Methanolic ammonia solutions but in xylene, which is used in the clean up procedure on the column.

All subsequent results were judged against a calibration graph constructed with these purified HMWA's. The metal coordination complexes of the naphthenic acids were acidified in acetic acid/xylene, and TLC and mass spectroscopy were performed on the resulting solution to show that the naphthenic acids were being complexed on a column.

TLC in an acidic mobile phase to break the complex to acids and on the acetic acid/xylene solution showed naphthenic acid spots compared to developed colors of Fluka naphthenic acids. These were compared to the acids from the parent material and to another North Sea naphthenic acid obtained from the respective solids from that field.

Low molecular weight acids were collected from the dialysate residue (DSR). A normal TAN titration and TLC were performed. Then the LMWA's were complexed on the column to construct a calibration graph.

All samples are cleaned up, dialyzed, and treated on the column. The UV spectrum is compared to the standard HMWA and LMWA graphs. A calculation is performed to relate back to the original sample. The HMWA's results are entered into a naphthenate probability index (NPI) calculation to give an index. This is correlated to known naphthenate fouling or to the non-fouling database of NPI.

The emulsion tendency can be similarly ranked versus the worst emulsion forming crude oil with the highest LMWA's known in practice.

Analyses indicate that naphthenic acids are distributed in the dialysis residues and the dialysate residues (DSR). The entire DR does not consist solely of naphthenic acids. Correcting for the naphthenic acids content then the non-acid content can be calculated. The latter has been found to have a wide range from 30 percent to 90 percent of the DR's. These non-acidic materials could be other HMW materials, insoluble naphthenate soaps, and hot hexane insoluble materials. Asphaltenes, resins, polymers, gums, and mineral matter are included here.

Thus analysis of the DR gives the HMW naphthenic acids which are corrosive in heat exchangers and capable of forming fouling soaps or acting as binders for the non acid constituents. The DSR gives the LMW naphthenic acids with potential for being distilled into the fractions causing emulsions, failure of fractions to pass quality control procedures.

### EXAMPLE 1

### Analysis of Crude Oil for Naphthenic Acid Content

Initially, the crude oils under test were stabilized gravimetrically utilizing nitrogen-purged heating to remove light ends greater than 70° C., and the water content was separated by the standard heated centrifuge method.

A stainless steel metal cage was constructed to hold the dialysis sack containing the sample and locked in position such that the sack does not expand to bursting. The dimensions of the metal cage were chosen such that several metal cages containing samples can be stacked to fit the one liter dialysis reservoir. This then meant that approximately eight oil samples could be dialyzed in one distillative reflux dialysis experiment. Samples were initially stabilized by gently heating up a known weight of sample under a nitrogen blanket to prevent oxidation. Each sample undergoes a heat-cool procedure to constant weight. The weight loss of volatiles is recorded and used in a correction procedure. The free water is removed by pipette or by a 60 seconds centrifuge spin (heated). The amount of removed free water (weight loss) is also used in a correction procedure when required.

The stabilized oils were then extracted by use of an exhaustive distillative-reflux membrane dialysis apparatus, and the sack retained non-dialyzable high molecular weight acids (HMWA), with a MW range of approximately 600 amu and above (including the naphthenate precursor ARN-type acids), which included any naphthenate soaps present. These would then be gravimetrically collected by removal of the extraction solvent by heating at 70° C., under a Nitrogen blanket. DR weight is recorded and converted to percentage DR, minus the sack weight, which is referred to as the dialysis residue (DR). The samples include solids, soap-emulsions, and sludges.

The solvent dialysate (DS) containing low molecular weight compounds including low molecular weight (LMW) naphthenic acids was then subjected to further analysis and the dialysis residue comprising the high molecular weigh (HMW) was not used further.

The DSR's were volumetrically prepared in normal organic solvents and eluted through a specially prepared copper ammonium macroreticular resin column using a series of chosen solvents (by experiments). This combination of solvents removes residual oil and asphaltenes/resins/waxes, leaving the low molecular weight naphthenic acids of interest, complexed (bound) on the column.

Copper is chosen here in the example, but transition metals give colored complexes from their oxidized states when they accept a ligand. Copper, Nickel and Molybdenum are safe to work with. Chromium is toxic so it is not used here. The transition metal is chemically bonded on to the macroreticular resin, which is then prepared to accept an amine/ammonium ligand. The macroreticular resin column is first converted to the transition metal form and coordinated with ammonia or amines from above. The ammonia/amines attach to the transition metal, and rapidly impart a characteristic blue color to the coordinated metal still attached to the resin. This complex now reacts with naphthenic acids to give colored complexes based on the amount of naphthenic acids attached to the metal. For example the cuprammonium ion does not give a UV spectra, but once complexed with naphthenic acids, a UV absorption is observed in the copper-carbonyl UV absorption area.

Xylene and heptane are used to clean up heavy oil residues if these are present in the DSR charged to the column. Xylene and toluene were found to be good solvents for these residues. IPA-water solvent clean up after the xylene wash, affords a swelling of the column to expand the pores of the resin to remove the residual xylene and thus the residual heavy residues if present. The sequential solvent washing was identified after lengthy trials, and it removes contaminants while leaving the acids of interest on the column. These coordinated metal complexed naphthenic acids may then be collected in two experimentally chosen elution solvents and carefully evaporated under nitrogen purge, slowly to dryness. The residues may then be combined, re-dissolved in an ammoniacal solution for spectrophotometric analysis.

Method of Sample Preparation

As above, the dialysis residue is collected and the hexane solvent is evaporated off and the percentage dialysis reside (% DR) is determined gravimetrically.

The dialysate comprises the hexane solubles and contains relatively lower molecular weight materials from generally 600 MW and less. The HMW materials and hexane insolubles are retained as the DR in the Dialysis membrane sack.

The Dialysate solution was set up in a distillation flask with thermometer and a nitrogen sparge, one bubble per second, and the hexane solvent was carefully distilled off at around 70° C. to leave approximately 50 milliliters solution of Dialysate Residue (DSR) in hexane.

The DSR solution was transferred to a 100 milliliter volumetric flask and volumetrically made up to 100 milliliters with hexane. This is the DSR stock solution. The weight in grams of DSR in the stock solution was calculated from the formula:

$$\frac{( ( 100 - \% DR ) ⨯ Weight grams of sample taken for dialysis )}{100}$$

The Column Method:

Based on the column dimensions described herein, sufficient aliquot of the DSR stock solution was taken to give approximately 500 mgs of DSR, and this was transferred to a measuring cylinder.

The column was prepared ready for accepting a sample as described herein. The aliquot of DSR solution is transferred to the column and the method is followed as described herein.

The complexes of LMWA's formed were collected in solution form as per the normal method, and the dried residues were re-dissolved and combined in methanolic ammonia for UV spectrophotometric analyses.

Preparation of the DSR of the Standard

A crude oil with known high emulsion characteristics was prepared and dialyzed as described above. The dialysate solution was collected as described above, and a stock solution of the DSR were made up in hexane.

Preparation of the Amberlyst 15 Column to Collect the Naphthenic Acids

An Amberlyst® A15 macroreticular resin column was prepared according to the following steps. The preparatory column was set up using column dimensions of 30 centimeters (one foot) resin bed×2.5 centimeters (one inch) inside diameter with a solvent reservoir head of 15 centimeters (six inches). The resin was washed with 100 milliliters of 5 percent hydrochloric acid solution. The resin was washed with approximately 100 milliliters distilled water, until the pH of the eluate was around pH 7. Next, 100 milliliters of 5% sodium hydroxide solution was added to the column and eluted at 1 milliliter per minute. The column was washed with water until the pH was near pH 7. The column was washed with approximately 100 milliliters of methanol. The column was washed with 100 milliliters of xylene. The column was washed with 100 milliliters of hexane-xylene solution in an 80-20 ratio.

Collection of LMW Naphthenic Acids for Calibration:

An aliquot was taken of the DSR of the standard crude to give 2.5 grams of DSR. This aliquot was added to the preparatory column. The hexane solvent was eluted at one milliliter per minute, until the eluates were near colorless. The column was eluted with a hexane-xylene 80-20 mixture until eluates were colorless. The column was eluted with a 5 percent solution of acetic acid in methanol until eluate was colorless. Approximately 150 milliliters of acetic acid-methanol solution were required. The methanolic-acetic acid solution was evaporated off under nitrogen until the vapors were neutral to litmus paper. The drying was finished off in an oven at 55° C. It was then cooled and dissolved in xylene, filtered through a Whatman No. 1 filter paper to remove sodium acetate. The filtrate of the naphthenic acids was collected in a weighed beaker. The evaporation of solvent was repeated and oven finished. The weight of naphthenic acids obtained was calculated, and the filtrate was dissolved in 100 milliliters of a hexane-xylene solvent in an 80-20 mix to give a stock LMW naphthenic acid solution.

Checking Integrity of the LMW Acids:

A TLC method was performed compared to Fluka and Aldrich Commercial naphthenic acids. To identify that naphthenic acids are present, a standard ASTM TAN D664 assay method was performed. An acid value test was used to detect naphthenic acids in the stock solution.

Preparation of Calibration Graph:

An aliquot of stock LMW naphthenic acid solution was taken and the complexation on the Cu metal removal rate (MRR) resin column was performed as per standard method. The standard method was followed with different aliquots of naphthenic acid solution. The UV absorbance was read at 218 nanometers+/−2 nanometers. A calibration graph was constructed.

Interpolation of Sample UV Absorbance to mg LMW Naphthenic Acids

From the gradient of the graph, one Absorbance unit at 218 nanometers was found to be equivalent to 0.6 milligrams of LMW naphthenic acids in final test solution.

### EXAMPLE 2

### Determination of the Ratio of High Molecular Weight to the Low Molecular Weight Naphthenic Acids in Crude Oil Samples

The example shows the evaluation of the fouling tendencies of two crude oils and the product streams from the pipestill.

The following samples were analyzed: 1. Crude Oil; 2. Crude Oil to Desalter (PS02); 3. Raw Jet Fuel; 4. Diesel Feed to E120; 5. Vacuum Gas Oil (VGO), VAC SS2 to catalytic feed hydrotreating unit (CFHU); 6. LGVO, VAC SIDE, STREAM 1 to Fluid Catalytic Cracking Unit (FCCU); 7. HAGO; and 8. Residual, vacuum pipe still bottoms-pitch.

The samples were subjected to four types of analyses in this part of the project for fouling and corrosion interpretations. The original samples were analyzed for TAN values using the normal ASTM D664 technique, then the bottoms residue (BR) was separated from the dialysate residue (DSR) and both fractions analyzed for high and low molecular weight naphthenic acids. The original samples were also analyzed for the total acids.

All results and observations are tabulated in the Results section. The data from the tables are graphically presented with interpretations of results alongside the graph. A discussion of the methodology is presented in the Discussion section.

Results

The results with interpretations and implications from the data obtained are as summarized as follows:

TABLE 1

| | Tests on Original | | | | | | | | BR | |
|---|---|---|---|---|---|---|---|---|---|---|
| | Sample | | | | DSR-N | DSR-N | BR | BR | ppm | Original |
| | TAN | % | % | BR-N | % | TAN | % | % | non-acids | NFI |
| Sample | D664 | BR | DSR | % HMWA | LMWA | D664 | Acids | Non-Acids | Normalized | (or NPI) |
| TK1704 | 3.11 | 12.1 | 88.9 | 3.91 | 2.8 | 2.06 | 32.2 | 67.8 | 82K | 12.7 |
| PS02 | 1.55 | 10.7 | 90.3 | 1.98 | 7 | 1.56 | 18.6 | 81.4 | 87K | 6.4 |
| Jet Fuel | 0.24 | 0.32 | 99.7 | 0.15 | 0.1 | 0.29 | 47.1 | 52.9 | 1693 | 3.2 |
| Diesel | 1.14 | 0.94 | 99.1 | 0.05 | 0.4 | 1.95 | 5.2 | 94.8 | 8911 | 0.9 |
| HAGO | 2.17 | 0.74 | 99.3 | 0.1 | 2.4 | 2.35 | 30.6 | 69.4 | 5136 | 1.2 |
| LVGO | 2.39 | 0.47 | 99.6 | 0.27 | 1.1 | 2.71 | 57.6 | 42.4 | 1993 | 7.8 |
| VGO | 2.68 | 5.63 | 94.3 | 5.62 | 2.6 | 1.69 | 36.3 | 63.7 | 36K | 6.6 |
| Residual | 5.7 | 36.4 | 63.7 | 9.69 | 0.4 | 0.55 | 26.5 | 73.4 | 26K | 30.2 |

Wherein BR is used to mean bottoms residue, LMWA is used to mean low molecular weight acids, DSR is used to mean dialysate residue, N is used to mean normalized data to original, HMWA is used to mean high molecular weight acids, Mod is used to mean modified, and NFI is used to mean naphthenate fouling index (equivalent to NPI).

The details of the results are shown in the following tables:

TABLE 2

Naphthenate Fouling Index (NFI)

| Ranking No | Sample | NFI |
|---|---|---|
| 1 | Residual | 30 |
| 2 | TK1704 | 12.7 |
| 3 | LVGO | 8 |
| 4 | VGO | 7 |
| 5 | PS02 | 6 |
| 6 | Jet fuel | 3 |
| 7 | PS19 HAGO | 1 |
| 8 | Diesel | 1 |

NOTE:
all fouling interpretations are based on high molecular weight naphthenic acids only

The Residual should be a known fouling fraction. All others compare against it. PS02 crude is two times lower fouling than TK1704 Crude. LVGO and VGO are fouling fractions and are relatively high. Fouling indices are compared to a naphthenic acid crude oil. PS19 HAGO & Diesel are relatively low fouling. An NFI fouling factor of seven is considered a high NFI. The HMWA's will act as binders to the non-HMWA's forming tight deposits in the HTEX's. A NFI value of two or below is considered to indicate a low probability for the occurrence of a fouling issue. The probability of a fouling issue to occur will increase with an increasing NFI.

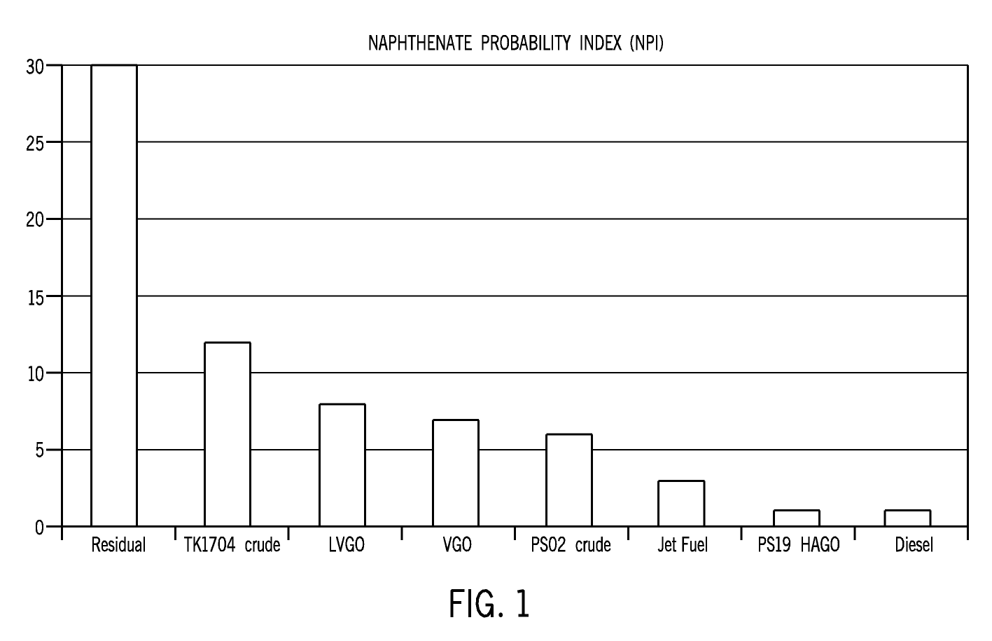

The content of Table 2 is represented graphically in FIG. 1.

The NFI increases in the correct order depending on the % HMWA's determined as set out in Table 3 below. TK 1704 crude has two times the fouling tendency compared to PS02 crude. The Jet Fuel has a moderate fouling tendency in heat exchangers.

TABLE 3
NAP Acid Distribution vs. TAN

| | NORMAL | | | |
|---|---|---|---|---|
| | TAN | % BR | % HWMA's | % LMWA's |
| CRUDE TK1704 | 3.11 | 12.10 | 3.91 | 2.8 |
| CRUDE PS02 | 1.56 | 10.70 | 1.98 | 7.0 |
| Jet Fuel (O) | 0.24 | 0.32 | 0.15 | 0.1 |
| DIESEL (O) | 1.12 | 0.94 | 0.05 | 0.4 |
| PS19-HAGO (O) | 2.17 | 0.74 | 0.1 | 2.4 |
| LVGO (O) | 2.39 | 0.47 | 0.27 | 1.1 |
| VGO (O) | 2.68 | 5.63 | 5.62 | 2.6 |
| Residual (O) | 5.70 | 36 | 9.69 | 0.4 |

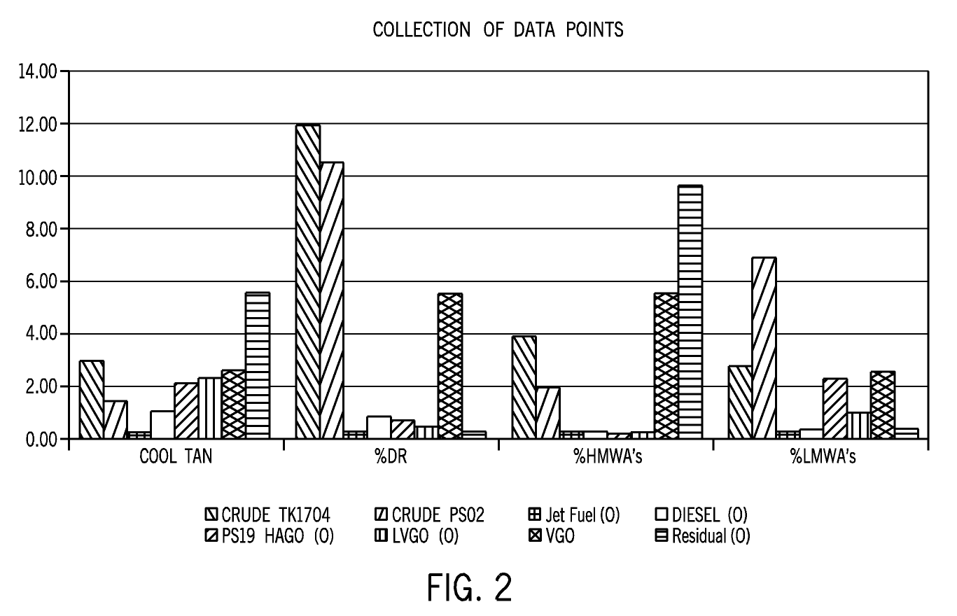

The content of Table 3 is represented graphically in FIG. 2.

Table 3 is a collection of important data points showing positive gradations in TAN's and % HMWA's as fractions come off at increasing temperatures. Similar series exist for BR's & % LMWA's in atmospheric section and another positive series for the vacuum section.

TABLE 4

| | BOTTOMS RESIDUES |
|---|---|
| | % Bottoms Residue |
| | (BR) |
| CRUDE TK1704 | 12.10 |
| CRUDE PS02 | 10.70 |
| Jet Fuel (O) | 0.32 |
| DIESEL (O) | 0.94 |
| PS19-HAGO (O) | 0.74 |
| LVGO (O) | 0.47 |
| VGO | 5.63 |
| Residual (O) | 36 |

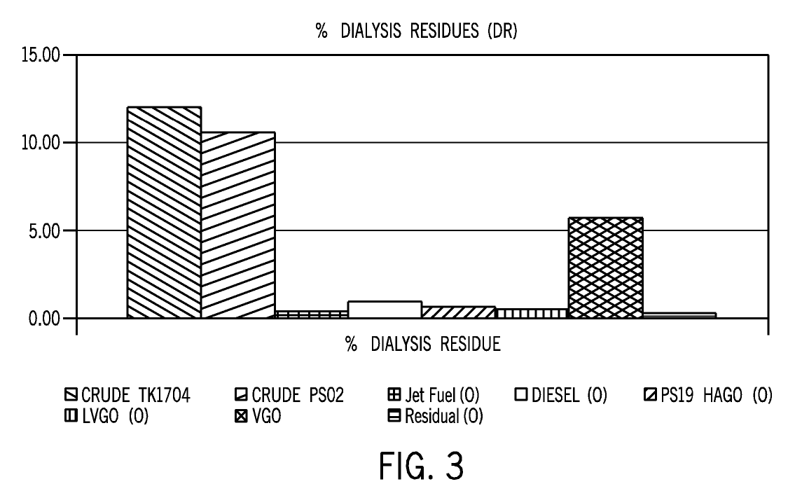

The content of Table 4 is represented graphically in FIG. 3.

Residual percentage BR (36 percent) is not included in the graph so that the graph can now show comparisons of relatively lower percentage BR values. Results show the two crude oils have similar percentage BR's. The BR's will consist of general hexane insoluble materials and compounds greater than 600 MW. HMWA's, napthenate soaps, asphaltenes, resins, gums, and particulate matter are expected in the BR's. The VGO has a high BR value and increases the probability of finding HMWA's.

TABLE 5

HMWA's v LMWA's

| | % HMWA's | % LMWA's |
|---|---|---|
| CRUDE TK1704 | 3.91 | 2.8 |
| CRUDE PS02 | 1.98 | 7.0 |
| Jet Fuel (O) | 0.15 | 0.1 |
| DIESEL (O) | 0.05 | 0.4 |
| PS19-HAGO (O) | 0.1 | 2.4 |
| LVGO (O) | 0.27 | 1.1 |
| VGO | 5.62 | 2.6 |
| Residual (O) | 9.69 | 0.3 |

(O) is normalized vs. the original sample

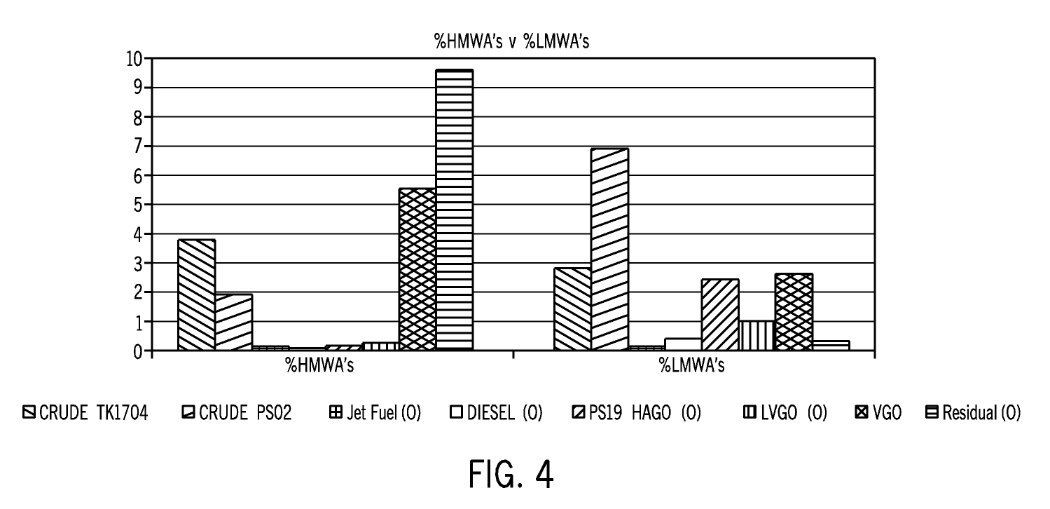

The content of Table 5 is represented graphically in FIG. 4.

The two crude oils are different in % HMWA's and % LMWA's. The % HMWA's increase as temperatures increase showing a high rate of increase to VGO (2.6 percent) and Resid fractions (9 percent). The % LMWA's also increase in that order in the atmospheric section but a second series increase to over 2.5 percent in the VGO. As expected the residual contains only 0.4 percent LMWA's but 9 percent HMWA's. As theorized, the higher the % LMWA's the lower the TAN compared to reverse observed for HMWA's.

TABLE 6
NORMAL TAN ASTM D664 VARIATION

| | NORMAL TAN |
|---|---|
| CRUDE TK1704 | 3.11 |
| CRUDE PS02 | 1.56 |
| Jet Fuel (O) | 0.24 |
| DIESEL (O) | 1.12 |
| PS19-HAGO (O) | 2.17 |
| LVGO (O) | 2.39 |
| VGO | 2.68 |
| Residual (O) | 5.70 |

(O) is normalized vs. the original sample

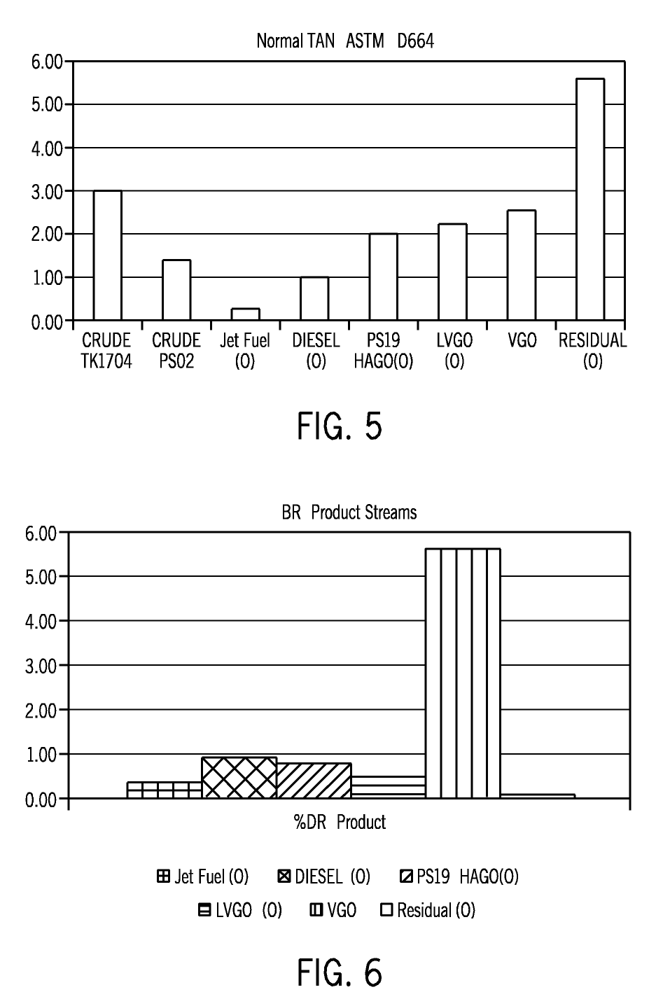

The content of Table 6 is represented graphically in FIG. 5.

The two crude oils show different TAN's by the normal method. TAN's increase as expected since temperatures increase in the Pipestill. Since LMWA's decrease as temperatures increase down the column, then the increases in TAN's are due to the HMWA's, which are implicated in corrosion and fouling tendencies.

TABLE 7

| | % BR Product |
|---|---|
| | Streams |
| CRUDE TK1704 | 12.10 |
| CRUDE PS02 | 10.70 |
| Jet Fuel (O) | 0.32 |
| DIESEL (O) | 0.94 |
| PS19-HAGO (O) | 0.74 |
| LVGO (O) | 0.47 |
| VGO | 5.63 |
| Residual (O) | 36.00 |

(O) is normalized vs. the original sample

The content of Table 7 is represented graphically in FIG. 6.

The % BR's increase three-fold from the Jet Fuel to the Diesel Fraction in the Atmospheric section, but increase rapidly in the vacuum section which is an expected result. Residual % BR (36 percent) is not plotted here to afford depth and clarity to the graph, since 36 percent would swamp the Y-axis.

TABLE 8

| | DSR TAN to |
|---|---|
| | Original |
| CRUDE TK1704 | 2.06 |
| CRUDE PS02 | 1.55 |
| Jet Fuel (O) | 0.29 |
| DIESEL (O) | 1.95 |
| PS19-HAGO (O) | 2.35 |
| LVGO (O) | 2.71 |
| VGO | 1.69 |
| Residual (O) | 0.55 |

(O) is normalized vs. the original sample

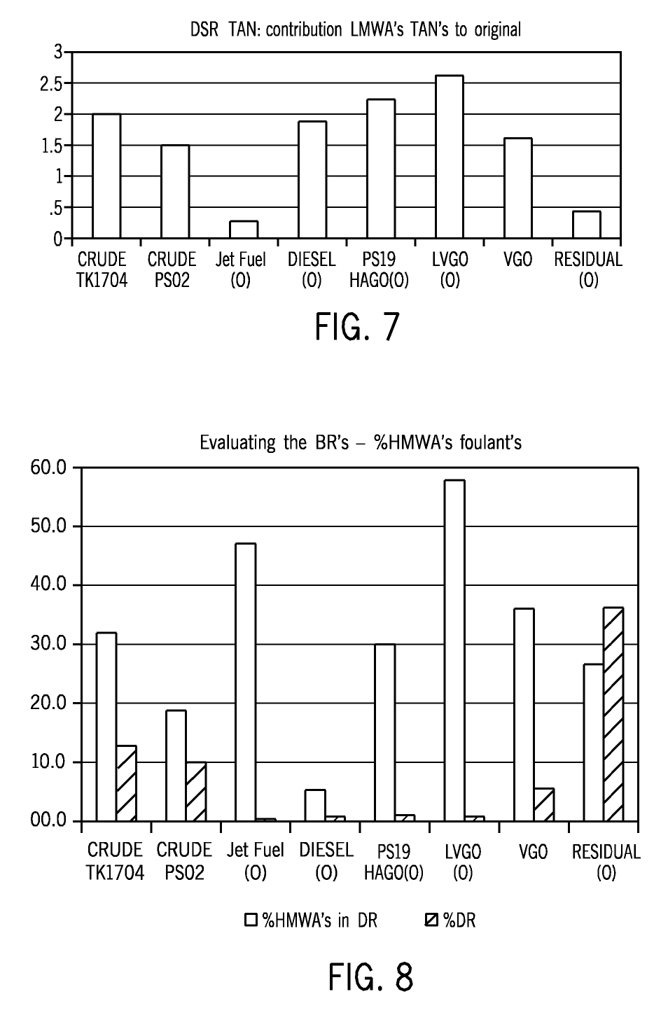

The content of Table 8 is represented graphically in FIG. 7.

There is a high contribution to the overall TAN's of the fractions from the LMWA's as the % DSR's on Atmospheric fractions and LVGO are in the 99 percent DSR's area. This decreased for the vacuum section of the Pipestill for VGO and Residual as LMWA's in these fractions are decreased. This is to be expected and it appears that HMWA's breaking up in the vacuum residual section does occur here, providing LMWA's to the VGO stream.

TABLE 9
Evaluating the BR's - % acid foulants

| | | | % Non-HMWA's in |
|---|---|---|---|
| Sample | % HMWA's in BR | % BR | BR |
| CRUDE TK1704 | 32.2 | 12.10 | 67.8 |
| CRUDE PS02 | 18.6 | 10.70 | 81.4 |
| Jet Fuel (O) | 47.1 | 0.32 | 52.9 |
| DIESEL (O) | 5.2 | 0.94 | 94.8 |
| PS19-HAGO (O) | 30.6 | 0.74 | 69.4 |
| LVGO (O) | 57.6 | 0.47 | 42.4 |
| VGO | 36.3 | 5.63 | 63.7 |
| Residual (O) | 26.5 | 36.00 | 73.5 |

(O) is normalized vs. the original sample

The content of Table 9 is represented graphically in FIG. 8.

PS02 crude has a lower (19 percent) % HMWA fouling material in BR compared to TK 1704 Crude (32 percent) in the BR's of these crudes. The BR's are essentially high molecular weight compounds and/or hexane insoluble naphthenic acid materials. All fractions show significant concentrations of non-HMWA's (40 percent to 90 percent) in the HMW BR's. These are most likely the soaps of Nap acids and/or materials physically transported up the column by foaming or velocity of fluids. HMWA's in VGO and Residual fluids will cause more fouling and could break up to LMWA's in follow-on processes leading to corrosion predictions.

TABLE 10
Non-Acidic components in the BR's

| | | | ppm potential |
|---|---|---|---|
| Sample | % Non-HMWA's in BR | % BR | foulants |
| CRUDE TK1704 | 81.4 | 12.10 | 98494 |
| CRUDE PSO2 | 67.8 | 10.70 | 72546 |
| Jet Fuel (O) | 52.9 | 0.32 | 1693 |
| DIESEL (O) | 94.8 | 0.94 | 8911 |
| PS19-HAGO (O) | 69.4 | 0.74 | 5136 |
| LVGO (O) | 42.4 | 0.47 | 1993 |
| VGO | 63.7 | 5.63 | 35863 |
| Residual (O) | 73.5 | 36.00 | 264600 |

(O) is normalized vs. the original sample

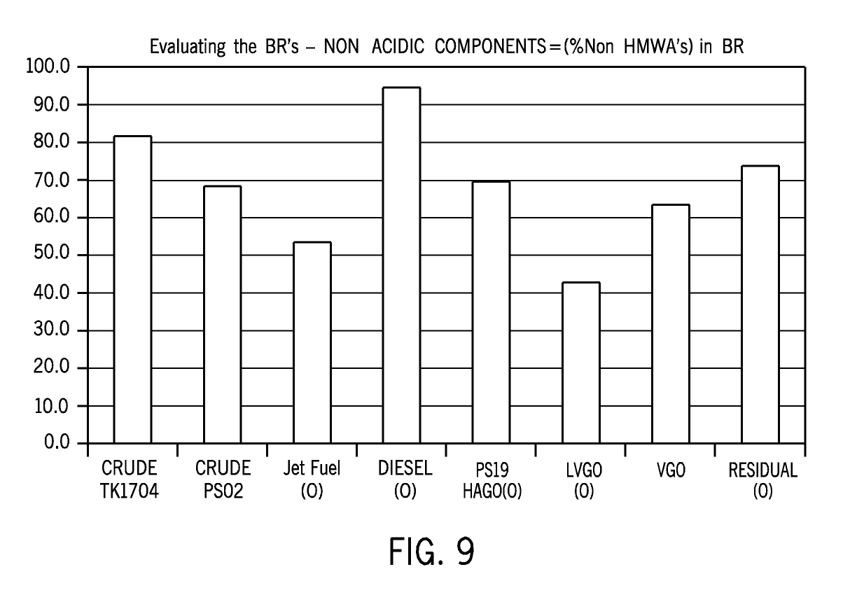

The content of Table 10 is represented graphically in FIG. 9.

All fractions show significant concentrations of non-HMWA's (40 percent to 90 percent) in the HMW BR's. These are most likely the soaps of Nap acids and/or materials physically transported up the column by foaming, surfactant effects or velocity of fluids. These can include gums, polymers, particulate matter, etc. In VGO, it is more likely to be dispersed resins, asphaltenes, and tars.

TABLE 11

| | BR results |
|---|---|
| | ppm potential |
| Sample | non-HMWA's foulants |
| CRUDE TK1704 | 98551 |
| CRUDE PS02 | 72521 |
| Jet Fuel (O) | 1693 |
| DIESEL (O) | 8915 |
| PS19-HAGO (O) | 5139 |
| LVGO (O) | 1993 |
| VGO | 35863 |
| Residual (O) | 264745 |

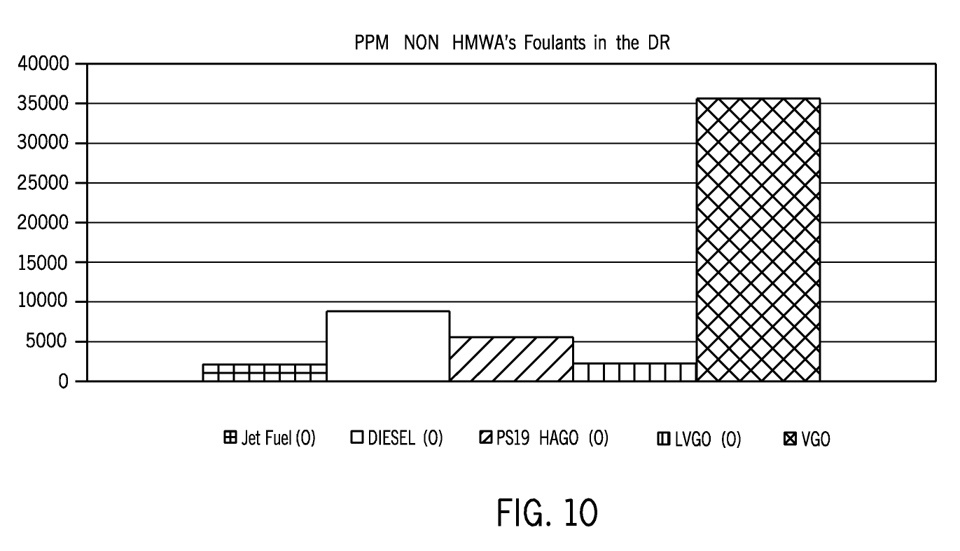

The content of Table 11 is represented graphically in FIG. 10.

The non-HMWA's foulants decrease from the Diesel fraction to the LVGO, but a very high concentration of extraneous foulant(s) is observed in the VGO, which correlates with the BR having a high value at 5 percent to 6 percent. Dispersed resins, tars and asphaltenes could be cause of high values here.

TABLE 12

| | ppm HMWA's |
|---|---|
| | foulants |
| Jet Fuel (O) | 1500 |
| DIESEL (O) | 500 |
| PS19-HAGO (O) | 1000 |
| LVGO (O) | 2700 |

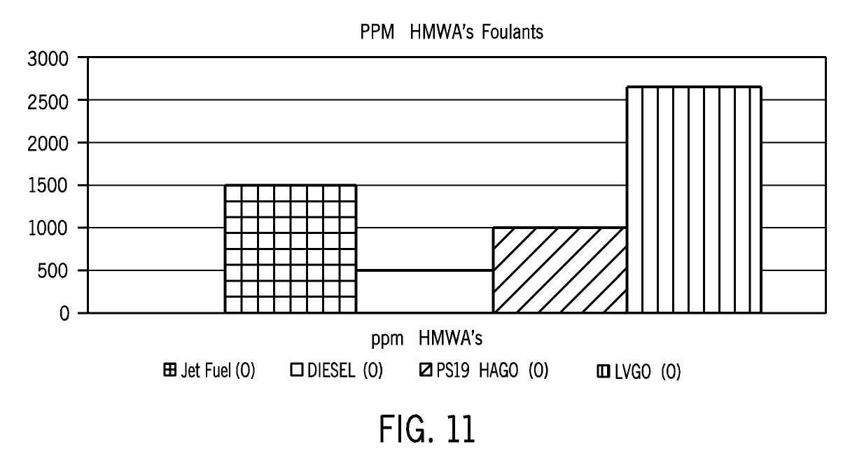

The content of Table 12 is represented graphically in FIG. 11.

The VGO is not included, as its ppm is very high (35K ppm). The ppm HMWA's foulants increase as temperatures increase in the Pipestill from diesel to LVGO and further on.

Discussion

All samples were cleaned up, separated and analyzed. The analysis results are compared to the standard HMWA and LMWA graphs. A calculation is performed to relate back to the original sample. The HMWA's results are entered into the NFI calculation to give an index. This is correlated to known naphthenate fouling or non-fouling database of NFI.

The emulsion tendency can be similarly ranked versus the worst emulsion forming crude oil with the highest LMWA's known in practice.

Analyses indicate that naphthenic acids are in the bottoms residues and the dialysate residues. The entire BR does not consist solely of naphthenic acids. Correcting for the naphthenic acids content then the non-acid content can be calculated. The latter ranged from 40 percent to 90 percent of the BR's. As indicated in the Conclusion section, these non-acids could be other HMW materials and hot hexane insoluble materials. Asphaltenes, resins, and mineral matter are included here.

Thus, analysis of the BR gives the HMW naphthenic acids, which are corrosive in heat exchangers and are capable of forming fouling soaps or acting as binders for the non-acid constituents. The DSR gives the LMW naphthenic acids with potential for being distilled into the fractions causing emulsions failure of fractions to pass quality control procedures.

Observations

1. TAN's in Relationship to HMWA's and LMWA's

The normal TAN method shows a general gradation in total naphthenic acid properties of the crude oils and product streams. It is noted that the percent high molecular weight acids are low in relative terms from the Jet Fuel, Diesel, LVGO, and HAGO fractions at less than 0.5% HMWA's.

The TAN's of the Jet Fuel to HAGO fractions are contributed mainly by the LMWA's contents 0.1 and 2.5% LMWA's respectively. VGO and Residual have the highest % HMWA's and the highest TAN's. The two crude oils, TK 1704 and PS02, are different in TAN's (3 and 1.6 TAN, respectively), and different in percent HMWA's content (4 percent and 2 percent approximately), and the two crude oils however also contrast each other in their % LMWA's content (3 and 7% LMWA's respectively).

2. Naphthenate Fouling Index NFI

The calculated Naphthenate Fouling Index (NFI), based on a database of fouling and non-fouling crudes, and high and low TAN's, indicates that the residual has the highest fouling index (31), and that there is a decreasing gradation from the Residual to the Jet Fuel. However, the NFI indicates LVGO and VGO to be high (8 and 7, respectively) fouling while Jet Fuel has an average medium fouling character. The two crude oils PS02 and TK 1704 are again different in the NFI (6 and 12 NFI, respectively). The HAGO and Diesel fractions are of low probability of fouling.

3. The Low Molecular Weight Materials in the Distillable Residues (DSR)—Relationship to Overall TAN Contribution

The DSR's of the fractions contribute their TAN's to the overall TAN values. This shows that the LMWA's are relatively medium-high in TAN values depending on the % DSR's and % LMWA's in these fractions. The VGO has a high LMWA's (2.6 percent) content, and thus a reasonable high contribution of TAN (1.7 TAN) to the original sample.

It is suggested that the HMWA's in the residual at 9 to 10% HMWA's are being fractured in the vacuum section to give the appropriate LMWA's of the correct boiling range to be distilled off with the VGO fraction. The two crude oils, TK1704 and PS02, have different TAN's (2.31 and −1.76, respectively) in their dialysate residues (DSR's) suggesting different percentage of molecular weight ranges of LMWA's.

4. The High Molecular Weight/Hexane Insoluble Materials in the Bottoms Residues (BR's)

The bottoms residues contain the high molecular weight acids, which are implicated in fouling and corrosion mechanisms. Results indicate that the crude oils, TK 1704 and PS02, have similar concentrations of these bottoms residues (12 percent and 11 percent, respectively). The bottoms residues gradually decrease in concentration from the Diesel to HAGO to LVGO fractions (0.9 percent, 0.7 percent, and 0.5 percent, respectively). The Jet Fuel has a 0.3 percent hexane insolubles in the BR, which is considered high for a jet fuel. The high molecular weight material in the BR's increase in the VGO and Residual samples (6 percent and 36 percent, respectively).

5. The High Molecular Weight Acids in the BR's

The crude oils, TK 1704 and PS02, although having comparable bottoms residue concentrations (12 percent and 11 percent, respectively), have % HMWA's that vary from 32 percent to 18 percent, respectively. Our technique overcomes the weakness of the pentane insolubles, which will not detect this diagnostic feature to fouling and corrosion potentials. All BR's of the fractions, except Diesel (5% HMWA's in BR) have high concentrations of HMWA's.

6. Non-High Molecular Weight Acid Foulants in the BR's

The non-HMWA's vary from 40 percent to 90 percent. Both of these results indicate not only a high potential to fouling as per the NFI above for naphthenic acids, but also inherent fouling potentials from the non-HMWA's and general corrosive characteristics.

The concentrations of non-HMWA's gradually decrease in values from the Diesel fraction to the LVGO (10K ppm to 2K ppm, respectively) but increase very sharply for the VGO fraction (35K ppm). Fouling by insoluble, high molecular weight materials, classed as non-HMWA's, is implicated for all fractions. In the BR of VGO, it is suggested that these materials are highly dispersed insoluble particulates of resins, tars, asphalts, mineral matter, etc. For the Residual, the non-acid materials in this fraction are the insoluble high molecular weight asphalts, bitumens, mineral matter, ash, etc.

7. Implications of the New Test in Evaluating the Residual Bottom of the Barrel Experiment

The new analytical procedure tells us that the residual contains valuable materials (64 percent), which are soluble in hot hexane. The hot hexane does not extract high molecular weight acids, and the acid value of the distillable residue is 0.55 TAN, a significant decrease from a nearly 6 TAN value.

The LMWA's in the residual is only 0.4 percent, and thus the major contributor to the overall acid contents is the high molecular weight acid, which is not soluble in hot aliphatic solvents. Evaluating this data, a process of hot solvent extraction can yield a workable feedstock leaving 30 percent to 40 percent for asphalts.

### CONCLUSIONS

The standard pentane insoluble test which would give similar values to our hexane insoluble test would not characterize that the two crude oils, TK 1704 and PS02, are dissimilar in respect to their problematic naphthenic acid contents.

The results indicate that PS1704 has twice the high molecular weight acid (HMWA) concentration in the BR than does PS02.

This fact indicates that TK1704 will possess higher fouling and corrosion tendencies than PS02 crude oil.

In contrast, the PS02 oil has 2.5 times more low molecular weight acids (LMWA's) than TK 1704, which definitely predicts that PS02 will cause more emulsion issues in desalters, poor desalting water quality, corrosion in units, and naphthenic acids in product distillates.

The proprietary Naphthenate Fouling Index (NFI) calculation is an index developed to predict the probability of a fouling issue to occur due to the deposition of naphthenate soaps. The NFI calculation predicts a gradation of indices from the worst fouling residual fraction to the least expected fouling, the diesel fraction. The NFI for the two crude oils, TK1704 and PS02, predicts that TK1704 has twice the probability of fouling HMWA's and soaps than does the PS02 crude. (Refer to Table 1.)

The method detects that fractions from the Jet Fuel to HAGO contain hundreds of ppm of HMWA's and extraneous insoluble non-acid materials. These materials should not be found in these fractions.

The test results can be diagnostic in troubleshooting refinery process problems with respect to fouling, corrosion versus physical conditions in a unit. The above tells us that a physical carryover is implicated in the movement of HMWA and insoluble non-acid materials up the column.

The results prove by logical extension to refinery technology that LMWA's should not be found in the residual fraction. Only 0.3% LMWA's is present in the residual but the VGO contains high % LMWA's. High molecular weight acids are being fractured in the residual to produce LMWA's for the VGO stream.

The presence of relatively high % HMWA's in the VGO and Residual means that one can interpret that these two fractions possess qualities for high fouling and corrosion in integrated refinery units and ancillary equipment handling and accepting the fractions.

The method suggests that, in a bottom of the barrel experiment, hot aliphatic solvent extraction of the Residual have 64 percent of organic materials, leaving 36 percent insoluble residuum containing asphaltenes, resins, high molecular weight acids/soaps, and mineral matter.

The pipestill side-cuts have all shown an amount of naphthenic acid contamination. The effects of these acids on the unit that receives these products needs to be considered. For example, the LVGO feedstock to the FCCU contains 0.3% HMWA and 1.1% LMWA. These naphthenic acids and their soaps will have implications at the heat exchangers, catalyst beds, and slurry oil separation tanks.

Although the foregoing description of the present invention has been shown and described with reference to particular embodiments and applications thereof, it has been presented for purposes of illustration and description and is not intended to be exhaustive or to limit the invention to the particular embodiments and applications disclosed. It will be apparent to those having ordinary skill in the art that a number of changes, modifications, variations, or alterations to the invention as described herein may be made, none of which depart from the spirit or scope of the present invention. The particular embodiments and applications were chosen and described to provide the best illustration of the principles of the invention and its practical application to thereby enable one of ordinary skill in the art to utilize the invention in various embodiments and with various modifications as are suited to the particular use contemplated. All such changes, modifications, variations, and alterations should therefore be seen as being within the scope of the present invention as determined by the appended claims when interpreted in accordance with the breadth to which they are fairly, legally, and equitably entitled.

### REFERENCES

1. Goldzal et al., 3rd Intl Conf. on Petroleum Phase Behaviour and Fouling, (2002), New Orleans, USA.
2. Rousseau et al., “Calcium Carbonate and Naphthenate Mixed Scale in Deep Offshore Fields,” SPE 68307 (2001).
3. Vinstad et al., “Fighting Naphthenate Deposition at the Heidrun Field,” (2003). SPE Oilfield Scale Symposium, SPE 80375.
4. Roden, J., “Calcium Naphthenate Control on the Blake Field,” proceedings from Flow Assurance Europe (IQPC 18th-19 Nov. 2003, London) (2003).
5. M. S. Turner & P. C. Smith, “Controls On Soap Scale Formation, Including Naphthenate Soaps—Drivers And Mitigation,” SPE Oilfield Scale Symposium, SPE 94339, Aberdeen, UK (2005).
6. Sorbie et al “Naphthenate Formation In Oil Production: General Theories And Field Observations,” RSC; Chemistry in the Oil Industry IX, Manchester, UK, 31 October-2 November, 2005.
7. Baugh et al., SPE 93011, SPE Intl Symposium on Oilfield Scale, Aberdeen, UK, 1-12 May, 2005.
8. Gallup, D. L., “Ca-Naphthenates and Metallic Soap Deposits: Formation and Mitigation,” IQPC—Flow Assurance—A Holistic Approach, December, 2004, Kuala Lumpur (2004).
9. Haynes, D., “Opportunities in Processing High TAN Crude,” presented at Hydrocarbon Asia (September 2002).
10. Medias et al., 5th SPE Oilfield Scale Symposium, SPE 80404, Aberdeen, UK (2003).
11. Acevedo et al., Energy Fuels 1999, 13, 333-335.
12. Lochte, H. L., & Littman, E. R., “The Petroleum Acids and Bases,” Chemical Publishing Co., Inc, NY (1955).
13. Seifert, W. K. & Howells, W. G., Anal. Chem. 1969, 41, 554-568.
14. Seifert, W. K. & Teeter, R. M., Anal. Chem. 1969, 41, 786-795.
15. Seifert, W. K, & Teeter R. M. Anal Chem. 1970, 42, 180-189
16. Oil Plus internal fluid characterisation reports (not published).
17. Naphthenic Acid Corrosion in Crude Distillation Units, Material Performance, 28, 1988, 32-43,
18. Goldzal et al., SPE 74661, SPE Intl Symposium on Oilfield Scale, Aberdeen, UK, 2002.
19. Brandl, O., PhD Thesis, Norwegian Inst. of Sci. and Tech, May 2005.
20. Derungs, W. A., Corrosion, 1956, 12, 41-46.
21. Babian-Kibala et al., Amer. Chem. Soc., 1998, 3, 106-110.
22. Roussis et al., (ExxonMobil) US20020086434—Jul. 4, 2002.
23. Green et al., High Resoluti. Chromatgr. 1994, 17, 427-438.
24. Havre et al., J Dispersion Sci. Techol., 2002.
25. McCarthy, R. & Duthie, A. J., J. Lipid. Res. 1962, 3, 117-119.
26. Havre, E. T, Colloid Polym. Sci. (2004), 282, 270-279.
27. Membrane Dialysis: determination of sugars in Dairy Products, Metrohm UK, Laboratory Talk, 2 Jul. 2004.
28. Costin, C. R., Rohm & Haas Company, PA, U.S. Pat. No. 4,076,622, 1975.
29. Tomczyk et al., Prepr. Am., Chem, Soc., Div, Pet, Chem., 1998, 3, 125, 123-125.
30. Brient J. A., Prepr. J. Amer. Chem. Soc., Div. Pet. Chem. 1998, 3, 131-133.

What is claimed is:

1. A method for the quantitation of the naphthenic acid content of molecular weight less than 600 Daltons in a hydrocarbon composition, comprising:
a. separating naphthenic acids of molecular weight less than 600 Daltons from the hydrocarbon composition;
b. applying the separated naphthenic acids of molecular weight of less than 600 Daltons to a macroreticular ion-exchange resin additionally comprising transition metal ions coordinated with an amine or ammonia in which the metal ion amine/ammonia coordinated complexes are bound to the surface of the resin;
c. washing the resin;
d. eluting the naphthenic acid-metal ion complexes from the resin; and
e. quantitating the number of naphthenic acid-metal ion complexes present in the eluate from the resin.

2. A method as defined in claim 1, wherein the hydrocarbon composition comprises crude oil.

3. A method as defined in claim 1, wherein step a. of separating naphthenic acid from the hydrocarbon composition is carried out by dialysis.

4. A method as defined in claim 1, wherein step a. of separating naphthenic acid from the hydrocarbon composition and step b. are combined by direct addition of the hydrocarbon composition to the resin.

5. A method as defined in claim 1, wherein the macro reticular ion-exchange resin comprises a transition metal ion selected from Copper (Cu), Silver (Ag), Nickel (Ni), Cobalt (Co), Iron (Fe), Manganese (Mn), Chromium (Cr), and Platinum (Pt).

6. A method as defined in claim 1, wherein the amine is of general formula: 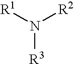
in which R¹, R², and R³ may each independently be Hydrogen (H), or a C₁-C₆ straight chain or branched alkyl group.

7. A method as defined in claim 6, wherein the amine is ethyl amine, ethylene diamine, morpholine, pyridine, cyclohexylamine, or benzylamine.

8. A method as defined in claim 1, wherein the washing of the resin in step c. is carried out using xylene, toluene, and/or heptane.

9. A method as defined in claim 8, wherein step c. is followed by a further wash using an isopropanol-water mixture.

10. A method as defined in claim 1, wherein eluting the naphthenic acid-metal ion complexes in step d. is carried out using an eluant selected from the group consisting of straight-chain or branched C₁-C₁, alkane, C₆-aryl, straight chain or branched C₁-C₆ alkyl alcohol or C₆-aryl alcohol, ammonia, and straight-chain or branched C₁-C₆ alkylamine.

11. A method as defined in claim 10, wherein the straight-chain or branched C₁-C₆ alkyl alcohol is methanol, ethanol, and/or isopropanol.

12. A method as defined in claim 10, wherein the C₁-C₁₀ alkane is n-heptane.

13. A method as defined in claim 10, wherein the straight chain or branched C₁-C₆ alkyl alcohol is an aqueous solution.

14. A method as defined in claim 10, wherein the straight chain or branched C₁-C₆ alkyl alcohol is an anhydrous preparation.

15. A method as defined in claim 10, wherein the straight chain or branched C₁-C₆ alkyl amine and/or ammonia is an aqueous solution.

16. A method as defined in claim 10, wherein the straight chain or branched C₁-C₆ alkyl amine and/or ammonia is formulated as a solution in a straight chain or branched C₁-C₆ alkyl alcohol.

17. A method as defined in claim 16, wherein the solution is methanolic ammonia.

18. A method as defined in claim 1, wherein quantitating the number of naphthenic acid-metal ion complexes present in the eluate is carried out by measuring the ultraviolet absorption of the complexes.

19. A method for the quantitation of the naphthenic acid content of molecular weight less than 600 Daltons in a hydrocarbon composition comprising crude oil, said method comprising:
a. separating naphthenic acids of molecular weight less than 600 Daltons from the hydrocarbon composition by dialysis;
b. applying the separated naphthenic acids of molecular weight of less than 600 Daltons to a macroreticular ion-exchange resin additionally comprising transition metal ions coordinated with an amine or ammonia in which metal ion amine/ammonia coordinated complexes are bound to the surface of the resin;
c. washing the resin using xylene, toluene, and/or heptane, or mixtures thereof;
d. washing the resin using an isopropanol-water mixture;
e. eluting the naphthenic acid-metal ion complexes from the resin using an eluant selected from the group consisting of straight-chain or branched C₁-C₁₀ alkane, C₆-aryl, straight chain or branched C₁-C₆ alkyl alcohol or C₆-aryl alcohol, ammonia, and straight-chain or branched C₁-C₆ alkylamine; and
f. quantitating the number of naphthenic acid-metal ion complexes present in the eluate from the resin by measuring the ultraviolet absorption of the complexes.

20. A method for the quantitation of the naphthenic acid content of molecular weight less than 600 Daltons in a hydrocarbon composition, comprising:
a. separating and purifying naphthenic acids of low molecular weight from the hydrocarbon composition;
b. applying the separated and purified naphthenic acids of low molecular weight to a column of macroreticular resin prepared in a transition metal form with a ligand;
c. washing the column with a heptane/xylene wash, or mixtures thereof, and subsequently washing it with an isopropanol-water mixture;
d. eluting the naphthenic acid-metal ion complexes from the resin with an alcoholic amine solution; and
e. quantitating the number of naphthenic acid-metal ion complexes present in the eluate from the resin.
